(19) United States
(12) Patent Application Publication
(10) Pub. No.: US 2006/0083715 A1
(43) Pub. Date: Apr. 20, 2006

(54) Interferon beta-like molecules for treatment of stroke

(76) Inventors: Steven Glazer, Copenhagen N (DK); Thomas Sager, Smoerum (DK)

Correspondence Address: MAXYGEN, INC.;INTELLECTUAL PROPERTY DEPARTMENT, 515 GALVESTON DRIVE, RED WOOD CITY, CA, 94063, US

(21) Appl. No.: 10/506,954

(22) PCT Filed: Feb. 28, 2003
(86) PCT No.: PCT/DK03/00127

§ 371 Date: Jun. 9, 2005

(30) Foreign Application Priority Data
Mar. 12, 2002 (DK) PA 2002 00371

(51) Int. Cl. A61K 38/21; C07K 14/565

(52) U.S. Cl. 424/85.6; 530/351

(57) ABSTRACT

The present invention relates to use of interferon beta-like polypeptides for treatment of stroke or transient ischemic attach in a primate, preferably in a human. More particularly, the interferon beta-like polypeptides differs from the amino acid sequence of wild-type human IFNB (SEQ ID NO:2) in that at least one glycosylation site, preferably at least one in vivo N-glycosyltaion site has been introduced. Optionally the interferon beta-like polypeptides are PEGylated.

### FIELD OF THE INVENTION

The present invention relates to use of interferon beta-like polypeptides for treatment of stroke or transient ischemic attack in a primate, preferably in a human.

### BACKGROUND OF THE INVENTION

Interferons are important cytokines characterized by antiviral, antiproliferative, and immunomodulatory activities. These activities form a basis for the clinical benefits that have been observed in a number of diseases, including hepatitis, various cancers and multiple sclerosis. The interferons are divided into the type I and type II classes. Interferon β (also designated interferon beta, IFNB or IFN-β) belongs to the class of type I interferons, which also includes interferon α, τ and ω, whereas interferon γ is the only known member of the distinct type II class.

Wild-type human IFNB is a regulatory polypeptide with a molecular weight of 22 kDa consisting of 166 amino acid residues. It can be produced by most cells in the body, in particular fibroblasts, in response to viral infection or exposure to other biologics. It binds to a multimeric cell surface receptor, and productive receptor binding results in a cascade of intracellular events leading to the expression of IFNB-inducible genes which in turn produces effects which can be classified as antiviral, antiproliferative and immunomodulatory.

The amino acid sequence of wild-type human IFNB was reported by Taniguchi, Gene 10:11-15, 1980, and in EP 0 083 069, EP 0 041 313 and U.S. Pat. No. 4,686,191.

Crystal structures have been reported for human and murine IFNB, respectively (Proc. Natl. Acad. Sci. USA 94:11813-11818, 1997; J. Mol. Biol. 253:187-207, 1995 and were reviewed in Cell Mol. Life Sci. 54:1203-1206, 1998).

Relatively few protein-engineered variants of IFNB have been reported (WO 95/25170; WO 98/48018; U.S. Pat. No. 5,545,723; U.S. Pat. No. 4,914,033; EP 0 260 350; U.S. Pat. No. 4,588,585; U.S. Pat. No. 4,769,233; Stewart et al., DNA Vol 6 no 2 1987 pp. 119-128 and Runkel et al., 1998, J. Biol. Chem. 273, No. 14, pp. 8003-8008).

Expression of IFNB in CHO cells has been reported (U.S. Pat. No. 4,966,843; U.S. Pat. No. 5,376,567 and U.S. Pat. No. 5,795,779).

Redlich et al., Proc. Natl. Acad. Sci., USA, Vol. 88, pp. 4040-4044, 1991 has described immunoreactivity of antibodies against synthetic peptides corresponding to peptide stretches of recombinant human IFNB with the mutation C17S.

IFNB molecules with a particular glycosylation pattern and methods for their preparation have been reported (EP 0 287 075 and EP 0 529 300).

Various references disclose modification of polypeptides by polymer conjugation or glycosylation. Polymer modification of native IFNB or a C17S variant thereof has been reported (EP 0 229 108; U.S. Pat. No. 5,382,657; EP 0 593 868; U.S. Pat. No. 4,917,888 and WO 99/55377).

U.S. Pat. No. 4,904,584 discloses PEGylated lysine-depleted polypeptides, wherein at least one lysine residue has been deleted or replaced with any other amino acid residue. WO 99/67291 discloses a process for conjugating a protein with PEG, wherein at least one amino acid residue on the protein is deleted and the protein is contacted with PEG under conditions sufficient to achieve conjugation to the protein. WO 99/03887 discloses PEGylated variants of polypeptides belonging to the growth hormone superfamily, wherein a cysteine residue has been susbstituted for a non-essential amino acid residue located in specified regions of the polypeptide. IFNB is mentioned as one example of a polypeptide belonging to the growth hormone superfamily. WO 00/23114 discloses glycosylated and PEGylated IFNB. IFNB fusion proteins are described in WO 00/23472.

Commercial preparations of IFNB are sold under the trade names Betaseron® (also termed interferon β1b, which is non-glycosylated, produced using recombinant bacterial cells, and which comprises the C17S mutation and has a deletion of the N-terminal methionine residue), Avonex® and Rebif® (also termed interferon β1a, which is glycosylated, produced using recombinant mammalian cells). These preparations are used for treatment of patients with multiple sclerosis, and have shown to be effective in reducing the exacerbation rate, and more patients remain exacerbation-free for prolonged periods of time as compared with placebo-treated patients. Furthermore, the accumulation rate of disability is reduced (Neurol. 51:682-689, 1998).

A comparison of interferon β1a and β1b with respect to structure and function has been presented in Pharmaceut. Res. 15:641-649, 1998.

IFNB is the first therapeutic intervention shown to delay the progression of multiple sclerosis, a relapsing then progressive inflammatory degenerative disease of the central nervous system. Its mechanism of action, however, remains largely unclear. It appears that IFNB has an inhibitory effect on the proliferation of leukocytes and antigen presentation. Furthermore, IFNB may modulate the profile of cytokine production towards an anti-inflammatory phenotype. Finally, IFNB can reduce T-cell migration by inhibiting the activity of T-cell matrix metalloproteases. These activities are likely to act in concert to account for the mechanism of IFNB in multiple sclerosis (Neurol. 51:682-689, 1998).

In addition, IFNB may be used for the treatment of osteosarcoma, basal cell carcinoma, cervical dysplasia, glioma, acute myeloid leukemia, multiple myeloma, Hodgkin's disease, breast carcinoma, melanoma, and viral infections such as papilloma virus, viral hepatitis, herpes genitalis, herpes zoster, herpetic keratitis, herpes simplex, viral encephalitis, cytomegalovirus pneumonia, and rhinovirus. Various side effects are associated with the use of current preparations of IFNB, including injection site reactions, fever, chills, myalgias, arthralgias, and other flu-like symptoms (Clin. Therapeutics, 19:883-893, 1997).

WO 01/15736 discloses novel IFNB conjugates comprising a non-polypeptide moiety attached to an IFNB polypeptide which have been modified by introduction and/or deletion of attachment sites for a non-polypeptide moiety, such as PEG, and glycosylation sites. The molecules have improved properties, such as improved half-life and/or reduced reactivity with neutralizing antibodies raised against current IFNB products.

Recently, IFNB has been suggested as a medicament in the treatment of stroke and related disorders (WO 01/41782; WO 02/089828; WO 02/080953 and Veldhuis et al. Stroke, January 2002, page 346).

### BRIEF DISCLOSURE OF THE INVENTION

The present invention provides IFNB-like polypeptides which are more efficient in the treatment of stroke and related disorders than is interferon β1a (e.g. Avonex® and Rebif®) and interferon β1b (e.g. Betaseron®).

Accordingly, in a first aspect the present invention relates to the use of an interferon β (IFNB) polypeptide variant comprising an amino acid sequence which differs from the amino acid sequence of wild-type human IFNB (SEQ ID NO:2) in that at least one glycosylation site has been introduced, for the manufacture of a medicament for the treatment of stroke or cerebrovascular accident (CVA) in a primate.

In another aspect the present invention relates to a method for treating or preventing stroke or cerebrovascular accident (CVA) in a primate, the method comprising administering an effective amount of an interferon β (IFNB) polypeptide variant comprising an amino acid sequence which differs from the amino acid sequence of wild-type human IFNB (SEQ ID NO:2) in that at least one glycosylation site has been introduced, to a primate in need thereof.

In a further aspect the present invention relates to the use of an interferon β (IFNB) polypeptide variant comprising an amino acid sequence which differs from the amino acid sequence of wild-type human IFNB (SEQ ID NO:2) in that at least one glycosylation site has been introduced, for the manufacture of a medicament for the treatment of transient ischemic attack in a primate.

In a still further aspect the present invention relates to a method for treating or preventing transient ischemic attack in a primate, the method comprising administering an effective amount of an interferon β (IFNB) polypeptide variant comprising an amino acid sequence which differs from the amino acid sequence of wild-type human IFNB (SEQ ID NO:2) in that at least one glycosylation site has been introduced, to a primate in need thereof.

Other aspects will be apparent from the below disclosure.

### DETAILED DISCLOSURE OF THE INVENTION

Definitions

In the context of the present application the following definitions apply:

The term “conjugate” (or interchangeably “conjugated polypeptide”) is intended to indicate a heterogeneous (in the sense of composite or chimeric) molecule formed by the covalent attachment of one or more polypeptide(s) to one or more non-polypeptide moieties. The term covalent attachment means that the polypeptide and the non-polypeptide moiety are either directly covalently joined to one another, or else are indirectly covalently joined to one another through an intervening moiety or moieties, such as a bridge, spacer, or linkage moiety or moieties using an attachment group present in the polypeptide. Preferably, the conjugate is soluble at relevant concentrations and conditions, i.e. soluble in physiological fluids such as blood. Examples of conjugated polypeptides for use in the invention include glycosylated and/or PEGylated polypeptides. The term “non-conjugated polypeptide” may be used about the polypeptide part of the conjugate.

The term “non-polypeptide moiety” is intended to indicate a molecule that is capable of conjugating to an attachment group of a polypeptide for use in the invention. Preferred examples of such molecules include polymer molecules, sugar moieties, lipophilic compounds, or organic derivatizing agents. When used in the context of a conjugate as described herein it will be understood that the non-polypeptide moiety is linked to the polypeptide part of the conjugate through an attachment group of the polypeptide.

The term “polymer molecule” is defined as a molecule formed by covalent linkage of two or more monomers, wherein none of the monomers is an amino acid residue, except where the polymer is human albumin or another abundant plasma protein. The term “polymer” may be used interchangeably with the term “polymer molecule”. Examples of preferred polymer molecules include PEG and MPEG. The term “polymer molecule” is also intended to cover carbohydrate molecules attached by in vitro glycosylation, i.e. synthetic glycosylation performed in vitro normally involving covalently linking a carbohydrate molecule to an attachment group of the polypeptide, optionally using a cross-linking agent.

Carbohydrate molecules attached by in vivo glycosylation, such as N- or O-glycosylation (as described further below) are referred to herein as “a sugar moiety”. Normally, the in vivo glycosylation site is an N-glycosylation site, but also an O-glycosylation site is contemplated as relevant for the present invention. It will be understood that a glycosylated IFNB variant may also be termed an IFNB conjugate (comprising a non-polypeptide moiety being a sugar moiety attached to the polypeptide part of the conjugate).

Except where the number of non-polypeptide moieties, such as polymer molecule(s) or sugar moieties in the conjugate is expressly indicated every reference to “a non-polypeptide moiety” contained in a conjugate or otherwise used herein shall be a reference to one or more non-polypeptide moieties, such as polymer molecule(s) or sugar moieties, in the conjugate.

The term “attachment group” is intended to indicate an amino acid residue group of the polypeptide capable of coupling to the relevant non-polypeptide moiety. For instance, for a polymer, in particular PEG, a frequently used attachment group is the ε-amino group of lysine or the N-terminal amino group. Other polymer attachment groups include a free carboxylic acid group (e.g. that of the C-terminal amino acid residue or of an aspartic acid or glutamic acid residue), suitably activated carbonyl groups, mercapto groups (e.g. that of a cysteine residue), aromatic acid residues (e.g. Phe, Tyr, Trp), hydroxy groups (e.g. that of Ser, Thr or OH-Lys), guanidine (e.g. Arg), imidazole (e.g. His), and oxidized carbohydrate moieties.

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ any amino acid residue that may or may not be identical to X′ and preferably is different from proline, N is asparagine and S/T/C is either serine, threonine or cysteine, preferably serine or threonine, and most preferably threonine). Although the asparagine residue of the N-glycosylation site is the one to which the sugar moiety is attached during glycosylation, such attachment cannot be achieved unless the other amino acid residues of the N-glycosylation site is present. Accordingly, when the non-polypeptide moiety is an N-linked sugar moiety, the term “amino acid residue comprising an attachment group for the non-polypeptide moiety” as used in connection with alterations of the amino acid sequence of the parent polypeptide is to be understood as amino acid residues constituting an N-glycosylation site is/are to be altered in such a manner that a functional N-glycosylation site is introduced into the amino acid sequence. For an “O-glycosylation site” the attachment group is the OH-group of a serine or threonine residue.

It should be understood that when the term “at least 25% (or 50%) of its side chain exposed to the solvent” is used in connection with introduction of an in vivo N-glycosylation site this term refers to the surface accessibility of the amino acid side chain in the position where the sugar moiety is actually attached. In many cases it will be necessary to introduce a serine or a threonine residue in position +2 relative to the asparagine residue to which the sugar moiety is actually attached and these positions, where the serine or threonine residues are introduced, are allowed to be buried, i.e. to have less than 25% (or 50%) of their side chains exposed to the solvent.

The sugar moiety attached to a glycosylation site is typically sialylated. However, the sialic acid may be removed, e.g. by enzymatic cleavage by neuramimidase, to produce an asialo-glycosylated IFNB polypeptide (Brady et al. J. Inher. Metab. Dis. (1994) 17, 510-519 and U.S. Pat. No. 5,549,892). In another embodiment, the sugar moieties are further modified to contain only mannose. This may be done by sequential treatment with neuramimidase, β-galactosidase and β-N-acetylglucosamimidase (Brady et al. J. Inher. Metab. Dis. (1994) 17, 510-519 and U.S. Pat. No. 5,549,892).

The term “amino acid residue comprising an attachment group for the non-polypeptide moiety” is intended to indicate that the amino acid residue is one to which the non-polypeptide moiety binds (in the case of an introduced amino acid residue) or would have bound (in the case of a removed amino acid residue).

The term “one difference” or “differs from” as used in connection with specific modifications, such as substitution, is intended to allow for additional differences being present apart from the specified amino acid difference. For instance, in addition to the removal and/or introduction of amino acid residues comprising an attachment group for the non-polypeptide moiety the IFNB polypeptide may comprise other substitutions that are not related to introduction and/or removal of such amino acid residues. These may, for example, include truncation of the C-terminus by one or more amino acid residues, truncation of the N-terminus by one or more amino acid residues and/or “conservative amino acid substitutions”, i.e. substitutions performed within groups of amino acids with similar characteristics, e.g. small amino acids, acidic amino acids, polar amino acids, basic amino acids, hydrophobic amino acids and aromatic amino acids. Examples of conservative substitutions in the present invention may in particular be selected from the groups listed in the table below.

| | | | | |
|---|---|---|---|---|
| 1 | Alanine (A) | Glycine (G) | Serine (S) | Threonine |
| | | | | (T) |
| 2 | Aspartic acid (D) | Glutamic acid (E) | | |
| 3 | Asparagine (N) | Glutamine (Q) | | |
| 4 | Arginine (R) | Histidine (H) | Lysine (K) | |
| 5 | Isoleucine (I) | Leucine (L) | Methionine (M) | Valine (V) |
| 6 | Phenylalanine (F) | Tyrosine (Y) | Tryptophan (W) | |

The term “at least one” as used about a non-polypeptide moiety, an amino acid residue, a substitution, etc. is intended to mean one or more.

In the present application, amino acid names and atom names (e.g. CA, CB, CD, CG, SG, NZ, N, O, C, etc) are used as defined by the Protein DataBank (PDB) (www.pdb.org) which are based on the IUPAC nomenclature (IUPAC Nomenclature and Symbolism for Amino Acids and Peptides (residue names, atom names e.t.c.), Eur. J. Biochem., 138, 9-37 (1984) together with their corrections in Eur. J. Biochem., 152, 1 (1985). CA is sometimes referred to as Cα, CB as Cβ. The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The terminology used for identifying amino acid positions/substitutions is illustrated as follows:

The terminology used for identifying amino acid positions/substitutions is illustrated as follows: C17 indicates that position 17 is occupied by a cysteine residue in the amino acid sequence shown in SEQ ID NO:2. C17S indicates that the Cys residue of position 17 has been replaced with a Ser residue. Multiple substitutions are indicated with a “+”, e.g. R71N+D73T/S means an amino acid sequence which comprises a substitution of the Arg residue in position 71 with an Asn residue and a substitution of the Asp residue in position 73 with a Thr or Ser residue, preferably a Thr residue. T/S as used about a given substitution herein means either a T or S residue, preferably a T residue. Deletions are indicated by an asterix. For example, M1* indicates that the Met residue in position 1 has been deleted. Insertions are indicated the following way: Insertion of an additional Phe residue after the Cys residue located in position 17 is indicated as C17CF. Combined substitutions and insertions are indicated in the following way: Substitution of the Cys residue at position 17 with an Ser residue and insertion of a Phe residue after the position 17 amino acid residue is indicated as C17SF.

The term “nucleotide sequence” is intended to indicate a consecutive stretch of two or more nucleotide molecules. The nucleotide sequence may be of genomic, cDNA, RNA, semisynthetic, synthetic origin, or any combinations thereof.

The term “IFNB protein sequence family” is used in its conventional meaning, i.e. to indicate a group of polypeptides with sufficiently homologous amino acid sequences to allow alignment of the sequences, e.g. using the CLUSTALW program. An IFNB sequence family is available, e.g. from the PFAM families, version 4.0, or may be prepared by use of a suitable computer program such as CLUSTALW version 1.74 using default parameters (Thompson et al., 1994, CLUSTAL W: improving the sensitivity of progressive multiple sequence alignment through sequence weighting, position-specific gap penalties and weight matrix choice, Nucleic Acids Research, 22:4673-4680).

“Cell”, “host cell”, “cell line” and “cell culture” are used interchangeably herein and all such terms should be understood to include progeny resulting from growth or culturing of a cell.

“Transformation” and “transfection” are used interchangeably to refer to the process of introducing DNA into a cell.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

The term “introduce” is primarily intended to mean substitution of an existing amino acid residue, but may also mean insertion of an additional amino acid residue.

The term “remove” is primarily intended to mean substitution of the amino acid residue to be removed by another amino acid residue, but may also mean deletion (without substitution) of the amino acid residue to be removed.

The term “modification”, as used herein, covers substitution, insertion and deletion.

The terms “mutation” and “substitution” are used interchangeably herein.

The term “immunogenicity” as used in connection with a given substance is intended to indicate the ability of the substance to induce a response from the immune system. The immune response may be a cell or antibody mediated response (see, e.g., Roitt: Essential Immunology (8^{th} Edition, Blackwell) for further definition of immunogenicity). Immunogenicity may be determined by use of any suitable method known in the art, e.g. in vivo or in vitro, e.g. using the in vitro immunogenicity test outlined in the Materials and Methods section below.

The term “reduced immunogenicity” as used about a given polypeptide or conjugate is intended to indicate that the conjugate or polypeptide gives rise to a measurably lower immune response than a reference molecule, such as wild-type human IFNB, e.g., Rebif® or Avonex®, or a variant of wild-type human IFNB, such as Betaseron®, as determined under comparable conditions. When reference is made herein to commercially available IFNB products (i.e. Betaseron®, Avonex® and Rebif®), it should be understood to mean either the formulated product or the IFNB polypeptide part of the product (as appropriate). Normally, reduced antibody reactivity (e.g. reactivity towards antibodies present in serum from patients treated with commercial IFNB products) is an indication of reduced immunogenicity.

The term “functional in vivo half-life” is used in its normal meaning, i.e. the time at which 50% of a given functionality of the polypeptide or conjugate is retained (such as the time at which 50% of the biological activity of the polypeptide or conjugate is still present in the body/target organ, or the time at which the activity of the polypeptide or conjugate is 50% of the initial value).

As an alternative to determining functional in vivo half-life, “serum half-life” may be determined, i.e. the time in which 50% of the polypeptide or conjugate molecules circulate in the plasma or bloodstream prior to being cleared. Determination of serum half-life is often more simple than determining functional in vivo half-life and the magnitude of serum half-life is usually a good indication of the magnitude of functional in vivo half-life. Alternative terms to serum half-life include “plasma half-life”, “circulating half-life”, “serum clearance”, “plasma clearance” and “clearance half-life”. The functionality to be retained is normally selected from antiviral, antiproliferative, immunomodulatory or receptor binding activity. Functional in vivo half-life and serum half-life may be determined by any suitable method known in the art as further discussed in the Materials and Methods section hereinafter.

The polypeptide or conjugate is normally cleared by the action of one or more of the reticuloendothelial systems (RES), kidney, spleen or liver, or by specific or unspecific proteolysis. Clearance taking place by the kidneys may also be referred to as “renal clearance” and is e.g. accomplished by glomerular filtration, tubular excretion or tubular elimination. Normally, clearance depends on physical characteristics of the polypeptide or conjugate, including molecular weight, size (diameter) (relative to the cut-off for glomerular filtration), charge, symmetry, shape/rigidity, attached carbohydrate chains, and the presence of cellular receptors for the protein. A molecular weight of about 67 kDa is considered to be an important cut-off-value for renal clearance.

Reduced renal clearance may be established by any suitable assay, e.g. an established in vivo assay. Typically, the renal clearance is determined by administering a labelled (e.g. radio-labelled or fluorescence-labelled) polypeptide or polypeptide conjugate to a patient and measuring the label activity in urine collected from the patient. Reduced renal clearance is determined relative to the corresponding non-conjugated polypeptide or the non-conjugated corresponding wild-type polypeptide or a commercial IFNB product under comparable conditions.

The term “increased” as used about the functional in vivo half-life or serum half-life is used to indicate that the relevant half-life of the conjugate or polypeptide is statistically significantly increased relative to that of a reference molecule, such as an un-conjugated wild-type human IFNB (e.g. Avonex® or Rebif®) or an un-conjugated variant human IFNB (e.g. Betaseron®) as determined under comparable conditions.

The term “reduced immunogenicity and/or increased functional in vivo half-life and/or increased serum half-life” is to be understood as covering any one, two or all of these properties. In an interesting embodiment, a conjugate or polypeptide as described herein has at least two of these properties, i.e. reduced immunogenicity and increased functional in vivo half-life, reduced immunogenicity and increased serum half-life or increased functional in vivo half-life and increased serum half-life.

The term “under comparable conditions” as used about measuring of relative (rather than absolute) properties of a molecule for use in the invention and a reference molecule is intended to indicate that the relevant property of the two molecules is assayed using the same assay (i.e. the assay is performed under the same conditions including the same internal standard), and, when relevant, the same type of animals.

The term “exhibiting IFNB activity” is intended to indicate that the polypeptide or conjugate has one or more of the functions of native IFNB, in particular human wild-type IFNB with the amino acid sequence shown in SEQ ID NO:2 (which is the mature sequence) optionally expressed in a glycosylating host cell, or any of the commercially available IFNB products. Such functions include capability to bind to an interferon receptor that is capable of binding IFNB and initiating intracellular signalling from the receptor, in particular a type I interferon receptor constituted by the receptor subunits IFNAR-2 and IFNAR-1 (Domanski et al., The Journal of Biological Chemistry, Vol. 273, No. 6, pp 3144-3147, 1998, Mogensen et al., Journal of Interferon and Cytokine Research, 19: 1069-1098, 1999), and antiviral, antiproliferative or immunomodulatory activity (which can be determined using assays known in the art (e.g. those cited in the following disclosure)). IFNB activity may be assayed by methods known in the art as exemplified in the Materials and Methods section hereinafter.

The polypeptide or conjugate “exhibiting” or “having” IFNB activity is considered to have such activity, when it displays a measurable function, e.g. a measurable receptor binding and stimulating activity (e.g. as determined by the primary or secondary assay described in the Materials and Methods section). The polypeptide exhibiting IFNB activity may also be termed “IFNB molecule”, “IFNB variant polypeptide” or “IFNB polypeptide” herein. The terms “IFNB polypeptide”, “IFNB variant” and “variant polypeptide” are primarily used herein about modified polypeptides for use in the invention.

The term “parent IFNB” is intended to indicate the starting molecule to be improved for use in accordance with the present invention. Preferably, the parent IFNB belongs to the IFNB sequence family. While the parent IFNB may be of any origin, such as vertebrate or mammalian origin (e.g. any of the origins defined in WO 00/23472), the parent IFNB is preferably wild-type human IFNB with the amino acid sequence shown in SEQ ID NO:2 or a variant thereof.

In the context of a parent IFNB polypeptide, a “variant” is a polypeptide, which differs in one or more amino acid residues from a parent IFNB polypeptide, such as wild-type human IFNB. Typically, the variant differs from the parent IFNB polypeptide, such as wild-type human IFNB, in 1-15 amino acid residues, 1-10 amino acid residues, 1-8 amino acid residues, 2-8 amino acid residues, 1-5 amino acid residues or 2-5 amino acid residues. Thus, typically the variant differs from the parent IFNB polypeptide, such as wild-type human IFNB, in 1, 2, 3, 4, 5, 6, 7, 8, 9, 10, 11, 12, 13, 14 or 15 amino acid residues. Examples of wild-type human IFNB polypeptides include the polypeptide part of Avonex® or Rebif®. An example of a parent IFNB variant is Betaseron®. Alternatively, the parent IFNB polypeptide may comprise an amino acid sequence, which is a hybrid molecule between IFNB and another homologous polypeptide, such as interferon α, optionally containing one or more additional substitutions introduced into the hybrid molecule. Such a hybrid molecule may contain an amino acid sequence, which differs in more than 10 amino acid residues from the amino acid sequence shown in SEQ ID NO:2. In order to be useful as a parent polypeptide the hybrid molecule exhibits IFNB activity (e.g. as determined in the secondary assay described in the Materials and Methods section herein). Other examples of variants of wild-type human IFNB that may serve as parent IFNB molecules in the present invention are the variants described in WO 01/15736 having introduced and/or removed amino acid residues comprising an attachment group for a non-polypeptide moiety, or any of the IFNB molecules described in WO 00/23114, WO 00/23472, WO 99/3887 or otherwise available in the art.

The term “functional site” as used about a polypeptide or conjugate for use in the invention is intended to indicate one or more amino acid residues which is/are essential for or otherwise involved in the function or performance of IFNB, and thus “located at” the functional site. The functional site is e.g. a receptor binding site and may be determined by methods known in the art, preferably by analysis of a structure of the polypeptide being complexed to a relevant receptor, such as the type I interferon receptor constituted by IFNAR-1 and IFNAR-2.

In the present context the term “increased glycosylation” is intended to indicate increased levels of attached carbohydrate molecules, normally obtained as a consequence of increased (or better) utilization of glycosylation site(s). The increased glycosylation may be determined by any suitable method known in the art for analyzing attached carbohydrate structures. One convenient assay for determining attached carbohydrate structures is the method described in Example 7 and 8 herein.

An amino acid residue “located close to” a glycosylation site is usually located in position −4, −3, −2, −1, +1, +2, +3 or +4 relative to the amino acid residue of the glycosylation site to which the sugar moiety is attached, in particular in position −2, −1, +1, or +2, such as position −1 or +1, in particular position −1. These positions may be modified to increase the glycosylation at the site. The modification is normally a substitution, the substitution being made with any other amino acid residue that gives rise to an increased glycosylation of the IFNB variant as compared to that of the parent IFNB polypeptide. Such other amino acid residue may be determined by trial and error type of experiments (i.e. by substitution of the amino acid residue of the relevant position to any other amino acid residue, and determination of the resulting glycosylation of the resulting variant).

When used herein the term “naturally occurring glycosylation site” is intended to mean the N-glycosylation site defined by N80 and T82.

When us used herein, the term “stroke” is intended to mean a condition resulting from the death of brain tissue resulting from the lack of blood flow and insufficient oxygen to the brain. The terms “stroke” and “cerebrovascular accident” (or “CVA”) are used interchangeably herein. As explained above, a stroke may be ischemic or hemorrhagic. Specific examples of ischemic stroke comprise embolic stroke, cardioembolic stroke, thrombotic stroke, large vessel thrombosis, lacunar infarction, artery-artery stroke and cryptogenic stroke. Specific examples of hemorrhagic stroke comprise subdural stroke, intraparenchymal stroke, epidural stroke and subarachnoid stroke.

In the present context, the term “transient ischemic attack” is intended to cover a disturbance in brain function resulting from a temporary deficiency in the brain's blood supply.

Variants for Use in the Invention

In a preferred embodiment of the invention the IFNB polypeptide is a variant of wild-type human IFNB, wherein said variant comprises at least one introduced (additional) in vivo glycosylation site. The introduced in vivo glycoylation site may be an O-glycosylation site, but is preferably an N-glycosylation site. In order to ensure that sugar moieties are attached to the glycosylation sites it will be understood that such glycosylated variants must be produced in a host cell capable of glycosylation.

Thus, in a preferred embodiment, the present invention relates to the use of an IFNB polypeptide variant comprising an amino acid sequence which differs from the amino acid sequence of wild-type human IFNB (SEQ ID NO:2) in that at least one in vivo N-glycosylation site has been introduced, for the manufacture of a medicament for the treatment of stroke or transient ischemic attack in a primate, preferably a human.

More particularly, the in vivo N-glycosylation site is introduced into a position of the parent IFNB molecule occupied by an amino acid residue exposed to the surface of the molecule, preferably with more than 25% of the side chain exposed to the solvent, in particular with more than 50% exposed to the solvent (these positions are identified in the Methods section herein). The in vivo N-glycosylation site is introduced in such a way that the N-residue of said site is located in said position. Analogously, an O-glycosylation site is introduced so that the S or T residue making up such site is located in said position. Furthermore, in order to ensure efficient glycosylation it is preferred that the in vivo glycosylation site, in particular the N residue of the N-glycosylation site or the S or T residue of the O-glycosylation site, is located within the first 141 amino acid residues of the IFNB polypeptide, more preferably within the first 116 amino acid residues.

Substitutions that lead to introduction of an additional in vivo N-glycosylation site at positions exposed at the surface of the parent IFNB molecule and occupied by amino acid residues having more than 25% of the side chain exposed to the solvent, include substitutions selected from the group consisting of

S2N+N4S/T, L6S/T, L5N+G7S/T, F8N+Q10S/T, L9N+R11S/T, R11N, R11N+S13T, S12N+N14S/T, F15N+C17S/T, Q16N+Q18S/T, Q18N+L20S/T, K19N+L21S/T, W22N+L24S/T, Q23N+H25S/T, G26N+L28S/T, R27N+E29S/T, L28S+Y30S/T, Y30N+L32S/T, L32N+D34S/T, K33N+R35S/T, R35N+N37S/T, M36N+F38S/T, D39S/T, D39N+P41S/T, E42N+I44S/T, Q43N+K45S/T, K45N+L47S/T, Q46N+Q48S/T, L47N+Q49T/S, Q48N+F50S/T, Q49N+Q51S/T, Q51N+E53S/T, K52N+D54S/T, L57N+I59S/T, Q64N+I66S/T, A68N+F70S/T, R71N+D73S/T, Q72N, Q72N+S74T, D73N, D73N+S75T, S75N+T77S, S75N, S76N+G78S/T, E81N+I83S/T, T82N+V84S/T, E85N+L87S/T, L88S/T, A89N+V91S/T, Y92S/T, Y92N+Q94S/T, H93N+195S/T, L98S/T, H97N+K99S/T, K99N+V101S/T, T100N+L102S/T, E103N+K105S/T, E104N+L106S/T, K105N+E107S/T, E107N+E109S/T, K108N+D110S/T, E109N+F111S/T, D110N+T112S, D110N, F111N+R113S/T, R113N+K115S/T, G114N+L116S/T, K115N+M117S/T, L116N, L116N+S118T, S119N+H212S/T, L120N+L122S/T, H121N+K123S/T, K123N+Y125S/T, R124N+Y126S/T, G127N+I129S/T, R128N+L130S/T, L130N+Y132S/T, H131N+L133S/T, K134N+K136S/T, A135N+E137S/T, K136N+Y138S/T, E137N, Y138N+H140S/T, H140N+A142S/T, V148N+I150S/T, R152N+F154S/T, Y155N+I157S/T, L160S/T, R159N+T161S, R159N, G162N+L164S/T and Y163N+R165S/T.

Substitutions that lead to introduction of an additional in vivo N-glycosylation site at positions exposed at the surface of the parent IFNB molecule having more than 50% of the side chain exposed to the solvent include substitutions selected from the group consisting of L6S/T, L5N+G7S/T, F8N+Q10S/T, L9N+R11S/T, S12N+N14S/T, F15N+C17S/T, Q16N+Q18S/T, K19N+L21S/T, W22N+L24S/T, Q23N+H25S/T, G26N+L28S/T, R27N+E29S/T, Y30N+L32S/T, K33N+R35S/T, R35N+N37S/T, M36N+F38S/T, D39S/T, D39N+P41 S/T, E42N+I44S/T, Q46N+Q48S/T, Q48N+F50S/T, Q49N+Q51S/T, Q51N+E53S/T, K52N+D54S/T, L57N+I59S/T, R71N+D73S/T, D73N, D73N+S75T, S75N+T77S, S75N, S76N+G78S/T, E81N+I83S/T, T82N+V84S/T, E85N+L87S/T, A89N+V91S/T, Y92S/T, Y92N+Q94S/T, H93N+I95S/T, T100N+L102S/T, E103N+K105S/T, E104N+L106S/T, E107N+E109S/T, K108N+D110S/T, D110N+T112S, D110N, F111N+R113S/T, R113N+K115S/T, L116N, L116N+S118T, K123N+Y125S/T, R124N+Y126S/T, G127N+I129S/T, H131N+L133S/T, K134N+K136S/T, A135N+E137S/T, E137N, V148N+I150S/T and Y155N+I157S/T.

Among the substitutions mentioned in the above lists, those are preferred that have the N residue introduced among the 141 N-terminal amino acid residues, in particular among the 116 N-terminal amino acid residues.

The presently most preferred substitutions include substitutions selected from the group consisting of

S2N+N4T/S, L9N+R11T/S, R11N, S12N+N14T/S, F15N+C17S/T, Q16N+Q18T/S, K19N+L21T/S, Q23N+H25T/S, G26N+L28T/S, R27N+E29T/S, L28N+Y30T/S, D39T/S, K45N+L47T/S, Q46N+Q48T/S, Q48N+F50T/S, Q49N+Q51T/S, Q51N+E53T/S, R71N+D73T/S, Q72N, D73N, S75N, S76N+G78T/S, L88T/S, Y92T/S, N93N+I95T/S, L98T/S, E103N+K105T/S, E104N+L106T/S, E107N+E109T/S, K108N+D110T/S, D110N, F111N+R113T/S and L116N, more preferably selected from the group consisting of S2N+N4T, L9N+R11T, Q49N+Q51T, R71N+D73T and F111N+R113T, even more preferably selected from the group consisting of Q49N+Q51T, R71N+D73T and F111N+R113T, in particular selected from the group consisting of Q49N+Q51T and F111N+R113T.

Specific examples of preferred IFNB variants include variants comprising substitutions selected from the group consisting of

Q49N+Q51T+F111N+R113T,

Q49N+Q51T+R71N+D73T+F111N+R113T,

S2N+N4T+F111N+R113T,

S2N+N4T+Q49N+Q51T,

S2N+N4T+Q49N+Q51T+F111N+R113T,

S2N+N4T+L9N+R11T+Q49N+Q51T,

S2N+N4T+L9N+R11T+F111N+R113T,

S2N+N4T+L9N+R11T+Q49N+Q51T+F111N+R113T,

L9N+R11T+Q49N+Q51T,

L9N+R11T+Q49N+Q51T+F111N+R113T or

L9N+R11T+F111N+R113T.

Most preferably, the IFNB variant comprises the substitutions Q49N+Q51T+F111N+R113T (leading to introduction of two additional in vivo N-glycosylation sites).

It will be understood that in order to introduce a functional in vivo N-glycosylation site the amino acid residue in between the N-residue and the S/T residue is different from a proline residue. Normally, the amino acid residue in between will be that occupying the relevant position in the amino acid sequence shown in SEQ ID NO:2. For instance, in the polypeptide comprising the substitutions Q49N+Q51T, position 50 is the position in between.

The IFNB variant may contain a single in vivo glycosylation site (e.g. the naturally occurring in vivo N-glycosylation site at N80). However, in order to obtain efficient shielding of epitopes present on the surface of the parent polypeptide it is often desirable that the polypeptide comprises more than one in vivo glycosylation site, in particular 2-7 or 2-5 in vivo glycosylation sites, such as 2, 3, 4, 5, 6 or 7 in vivo glycosylation sites. Thus, the IFNB polypeptide may comprise one additional glycosylation site (in addition to the naturally occurring in vivo N-glycosylation site already present at position N80), or may comprise 1-6 or 1-4 additional (introduced) in vivo glycosylation sites, such as 1, 2, 3, 4, 5, or 6 additional (introduced) in vivo glycosylation sites. Preferably, said in vivo glycosylation sites are in vivo N-glycosylation sites. Accordingly, the IFNB variant may contain one sugar moiety (e.g. the naturally occurring sugar moiety present at N80), but it is desirable that the IFNB variant comprises more than one sugar moiety, in particular 2-7 or 2-5 sugar moieties, such as 2, 3, 4, 5, 6 or 7 sugar moieties.

In a highly preferred embodiment, the IFNB polypeptide comprises three in vivo N-glycosylation sites (i.e. two additional (introduced) in vivo N-glycosylation sites (in addition to the naturally occurring N80 N-glycosylation site)), i.e. the IFNB variant comprises three in vivo N-glycosylation sites and three sugar moieties. In a particular preferred embodiment, the three in vivo N-glycosylation sites are located in positions 49, 80 and 111.

Further Modifications

Any of the above-disclosed glycosylated variants may be further modified.

For example, it is presently preferred that the IFNB polypeptide is free from a cysteine residue, e.g. the cysteine residue located in position 17 of SEQ ID NO:2. Preferably, the cysteine residue has been removed by the substitution C17S.

Accordingly, in a preferred embodiment, the present invention relates to the use of an IFNB polypeptide variant comprising an amino acid sequence which differs from the amino acid sequence of wild-type human IFNB (SEQ ID NO:2) in that at least one in vivo N-glycosylation site has been introduced and wherein the cyteine residue located at position 17 has been removed, for the manufacture of a medicament for the treatment of stroke or transient ischemic attack in a primate, preferably a human. Preferably, said cysteine residue has been removed by the substitution C17S.

Specific examples of particular preferred IFNB variants include variants comprising substitutions selected from the group consisting of

C17S+Q49N+Q51T,

C17S+F111N+R113T,

C17S+Q49N+Q51T+F111N+R113T,

C17S+Q49N+Q51T+R71N+D73T+F111N+R113T,

S2N+N4T+C17S+F111N+R113T,

S2N+N4T+C17S+Q49N+Q51T,

S2N+N4T+C17S+Q49N+Q51T+F111N+R113T,

S2N+N4T+L9N+R11T+C17S+Q49N+Q51T,

S2N+N4T+L9N+R11T+C17S+F111N+R113T,

S2N+N4T+L9N+R11T+C17S+Q49N+Q51T+F111N+R113T,

L9N+R11T+C17S+Q49N+Q51T,

L9N+R11T+C17S+Q49N+Q51T+F111N+R113T and

L9N+R11T+C17S+F111N+R113T.

Most preferably, the IFNB variant comprises the substitutions C17S+Q49N+Q51T+F111N+R113T (leading to introduction of two additional in vivo N-glycosylation sites at positions 49 and 111, and removal of the cysteine residue at position 17).

In a further preferred embodiment, the IFNB variant further comprises one or more substitutions located close to a glycosylation site in order to optimize or increase the glycosylation at the site. Specific examples are described in the section entitled “Variants with increased glycosylation”, pp. 14-23, in WO 02/074806.

In an interesting embodiment of the invention, the IFNB variant comprises an amino acid substitution in position 48, in particular if the variant comprises an introduced in vivo N-glycosylation site in position 49. Preferably, the glutamine residue located at position 49 is substituted with a hydrophobic amino acid residue, such as Q48F, Q48V, Q48W or Q48Y.

In a highly preferred embodiment of the invention, the IFNB variant comprises an amino acid substitution in position 110, in particular if the variant comprises an introduced in vivo N-glycosylation site in position 111. Preferably, the aspartic acid residue located at position 110 is substituted with a hydrophobic amino acid residue, such as D110F, D110V, D110W or D110Y. In a particular preferred embodiment the variant comprises the substitution D110F, preferably in combination with the substitutions F111N+R113T/S, in particular F111N+R113T

Accordingly, specific examples of particular preferred IFNB variants include variants comprising substitutions selected from the group consisting of

D110F+F111N+R113T,

Q49N+Q51T+D110F+F111N+R113T,

Q49N+Q51T+R71N+D73T+D110F+F111N+R113T,

S2N+N4T+D110F+F111N+R113T,

S2N+N4T+Q49N+Q51T+D110F+F111N+R113T,

S2N+N4T+L9N+R11T+D110F+F111N+R113T,

S2N+N4T+L9N+R11T+Q49N+Q51T+D110F+F111N+R113T,

L9N+R11T+Q49N+Q51T+D110F+F111N+R113T and

L9N+R11T+D110F+F111N+R113T.

Even more preferably, the IFNB variant comprises substitutions selected from the group consisting of

C17S+D110F+F111N+R113T,

C17S+Q49N+Q51T+D110F+F111N+R113T,

C17S+Q49N+Q51T+R71N+D73T+D110F+F111N+R113T,

S2N+N4T+C17S+D110F+F111N+R113T,

S2N+N4T+C17S+Q49N+Q51T+D110F+F111N+R113T,

S2N+N4T+L9N+R11T+C17S+D110F+F111N+R113T,

S2N+N4T+L9N+R11T+C17S+Q49N+Q51T+D110F+F111N+R113T,

L9N+R11T+C17S+Q49N+Q51T+D110F+F111N+R13T and

L9N+R11T+C17S+D110F+F111N+R113T.

Most preferably, the IFNB variant comprises the substitutions C17S+Q49N+Q51T+D110F+F111N+R113T(SEQ ID NO:3).

Conjugation

The glycosylated variants disclosed above may be further conjugated to a non-polypeptide moiety which is different from a sugar moiety. Specific examples are disclosed in WO 01/15736 in the sections entitled “Conjugate of the invention, wherein the non-polypeptide moiety is a molecule that has lysine as an attachment group” (pp. 17-22), “Conjugate of the invention wherein the non-polypeptide moiety binds to a cysteine residue” (pp. 22-23) and “Conjugate of the invention wherein the non-polypeptide moiety binds to an acid group” (pp. 23-25).

By removing and/or introducing amino acid residues comprising an attachment group for the non-polypeptide moiety it is possible to specifically adapt the polypeptide so as to make the molecule more susceptible to conjugation to the non-polypeptide moiety of choice, to optimize the conjugation pattern (e.g. to ensure an optimal distribution of non-polypeptide moieties on the surface of the IFNB molecule and thereby, e.g., effectively shield epitopes and other surface parts of the polypeptide without significantly impairing the function thereof). For instance, by introduction of attachment groups, the IFNB polypeptide is boosted or otherwise altered in the content of the specific amino acid residues to which the relevant non-polypeptide moiety binds, whereby a more efficient, specific and/or extensive conjugation is achieved. By removal of one or more attachment groups it is possible to avoid conjugation to the non-polypeptide moiety in parts of the polypeptide in which such conjugation is disadvantageous, e.g. to an amino acid residue located at or near a functional site of the polypeptide (since conjugation at such a site may result in inactivation or reduced IFNB activity of the resulting conjugate due to impaired receptor recognition). Further, it may be advantageous to remove an attachment group located closely to another attachment group in order to avoid heterogeneous conjugation to such groups.

It will be understood that the amino acid residue comprising an attachment group for a non-polypeptide moiety, either it be removed or introduced, is selected on the basis of the nature of the non-polypeptide moiety and, in most instances, on the basis of the conjugation method to be used. For instance, when the non-polypeptide moiety is a polymer molecule, such as a polyethylene glycol or polyalkylene oxide derived molecule, amino acid residues capable of functioning as an attachment group may be selected from the group consisting of lysine, cysteine, aspartic acid, glutamic acid and arginine. When the non-polypeptide moiety is a sugar moiety the attachment group is an in vivo glycosylation site, preferably an N-glycosylation site. Whenever an attachment group for a non-polypeptide moiety is to be introduced into or removed from the IFNB polypeptide, the position of the IFNB polypeptide to be modified is conveniently selected as follows:

The position is preferably located at the surface of the IFNB polypeptide, and more preferably occupied by an amino acid residue that has more than 25% of its side chain exposed to the solvent, preferably more than 50% of its side chain exposed to the solvent. Such positions have been identified on the basis of an analysis of a 3D structure of the wild-type human IFNB molecule as described in the Methods section herein.

Functional in vivo half-life is inter alia dependent on the molecular weight of the conjugate and the number of attachment groups needed for providing increased half-life thus depends on the molecular weight of the non-polypeptide moiety in question. In one embodiment, the conjugate for use in the invention has a molecular weight of at least 67 kDa, in particular at least 70 kDa as measured by SDS-PAGE according to Laemmli, U.K., Nature Vol 227 (1970), p 680-85. IFNB has a molecular weight of about 20 kDa, and therefore additional about 50 kDa is required to obtain the desired effect. This may be, e.g., be provided by 5, 10, 12, or 20 kDa PEG molecules or as otherwise described herein.

In a further embodiment the conjugate for use in the invention has one or more of the following improved properties (determined under comparable conditions):

Reduced immunogenicity as compared to wild-type human IFNB (e.g. Avonex® or Rebif®) or to Betaseron®, such as a reduction of at least 25%, more preferably at least 50%, and even more preferably at least 75%;

Increased functional in vivo half-life and/or increased serum half-life as compared to wild-type human IFNB (e.g. Avonex® or Rebif®) or to Betaseron®;

Reduced or no reaction with neutralizing antibodies from patients treated with wild-type human IFNB (e.g. Rebif® or Avonex®) or with Betaseron®, e.g. a reduction of neutralisation of at least 25%, such as of at least 50%, and preferably of at least 75% as compared to the wild-type human IFNB (e.g. Rebif® or Avonex®) or Betaseron®.

The magnitude of the antiviral activity of a conjugate for use in the invention may not be critical, and thus be reduced (e.g. by up to 75%) or increased (e.g. by at least 5%) or equal to that of wild-type human IFNB ((e.g. Avonex® or Rebif®) or to Betaseron® as determined under comparable conditions.

Furthermore, the degree of antiviral activity as compared to antiproliferative activity of a conjugate for use in the invention may vary, and thus be higher, lower or equal to that of wild-type human IFNB.

The non-polypeptide moiety is preferably a polymer molecule, such as PEG, and the polymer is covalently attached to an amino acid residue of the variant where the amino acid residue comprises an attachment group for the polymer molecule. Examples of such attachment groups are shown in the table on page 7-8 in WO 03/002152. Preferred attachment groups include the N-terminal amino group, the ε-amino group of a lysine residue and the —S—H group of a cysteine residue, in particular the N-terminal amino group and the ε-amino group of a lysine residue.

In a preferred embodiment at least one lysine residues of the parent polypeptide has been removed, e.g. by any of the substitutions mentioned in the section entitled “Conjugate of the invention, wherein the non-polypeptide moiety is a molecule which has lysine as an attachment group”, pp. 17-23, in WO 01/15736.

Thus, in one embodiment of this aspect of the invention the amino acid sequence of the IFNB variant differs from that of human wild-type IFNB in at least one lysine residue has been removed. Typically 1-5 lysine residues have been removed, in particular 14 or 1-3 lysine residues have been removed. The lysine residue(s) to be removed, preferably by substitution, is selected from the group consisting of K19, K33, K45, K52, K99, K105, K108, K115, K123, K134, and K136, preferably K19, K33, K45 and K123. The lysine residue(s) may be replaced with any other amino acid residue, but is preferably replaced by an arginine or a glutamine residue in order to give rise to the least structural difference.

Accordingly, the IFNB variants disclosed herein may contain further substitutions selected from the group consisting of

K19R, K33R, K45R, K123R, K19R+K33R, K19R+K45R, K19R+K123R, K33R+K45R, K33R+K123R, K45R+K123R, K19R+K45R+K123R, K19R+K33R+K123R, K19R+K33R+K45R, K33R+K45R+K123R and K19R+K33R+K45R+K123R, preferably K19R+K45R+K123R, K19R+K33R+K123R, K19R+K33R+K45R and K33R+K45R+K123R, in particular K19R+K33R+K45R.

Thus, specific examples of preferred IFNB conjugates to which at least one non-polypeptide moiety, such as a polymer molecule, in particular PEG, is covalently attached to an attachment group of an amino acid residue of the variant, include IFNB variants comprising substitutions selected from the group consisting of

C17S+Q49N+Q51T+K19R+K33R+K45R,

C17S+D110F+F111N+R113T+K19R+K33R+K45R,

C17S+Q49N+Q51T+D110F+F111N+R113T+K19R+K33R+K45R,

C17S+Q49N+Q51T+R71N+D73T+D110F+F111N+R113T+K19R+K33R+K45R,

S2N+N4T+C17S+D110F+F111N+R113T+K19R+K33R+K45R,

S2N+N4T+C17S+Q49N+Q51T+D110F+F111N+R113T+K19R+K33R+K45R,

S2N+N4T+L9N+R11T+C17S+D110F+F111N+R113T+K19R+K33R+K45R,

S2N+N4T+L9N+R11T+C17S+Q49N+Q51T+D110F+F111N+R113T+K19R+K33R+K45R,

L9N+R11T+C17S+Q49N+Q51T+D110F+F111N+R113T+K19R+K33R+K45R and

L9N+R11T+C17S+D110F+F111N+R113T+K19R+K33R+K45R, in particular

C17S+Q49N+Q51T+D110F+F111N+R113T+K19R+K33R+K45R.

When the IFNB variant is PEGylated it usually comprises 1-5 polyethylene glycol (PEG) molecules. In a further embodiment the IFNB molecule comprises 1-5 PEG molecules, such as 1-3 PEG molecules, e.g. 1, 2 or 3 PEG molecules. In a further embodiment each PEG molecule has a molecular weight of about 5 kDa (kilo Dalton) to 100 kDa, such as a molecular weight of about 10 kDa to 40 kDa, e.g. about 12 kDa or about 20 kDa. In a particular preferred embodiment of the invention the IFNB variant comprises 1 PEG molecule having a molecular weight of about 20 kDa.

When used about polymer molecules herein, the word “about” indicates an approximate average molecular weight and reflects the fact that there will normally be a certain molecular weight distribution in a given polymer preparation.

Suitable PEG molecules are available from Shearwater Polymers, Inc. and Enzon, Inc. and may be selected from SS-PEG, NPC-PEG, aldehyd-PEG, mPEG-SPA, mPEG-SCM, mPEG-BTC, SC-PEG, tresylated mPEG (U.S. Pat. No. 5,880,255), or oxycarbonyl-oxy-N-dicarboxyimide-PEG (U.S. Pat. No. 5,122,614).

Methods of Preparing a Conjugate

Specific details concerning conjugation of non-polypeptide moieties, in particular PEG polymers, to the IFNB variants disclosed herein are given in the section entitled “Methods of preparing a conjugate of the invention”, pp. 32-40, in WO 01/15736

Coupling to a Sugar Moiety

In order to achieve in vivo glycosylation of an IFNB polypeptide as described herein, the nucleotide sequence encoding the IFNB variant must be inserted in a glycosylating, eucaryotic expression host, such as an CHO cell. Suitable expression host cells are described in the section entitled “coupling to a sugar moiety”, p. 36, in WO 01/15736.

Methods of Preparing an IFNB Polypeptide Variant

The IFNB variants for use in the present invention, optionally in glycosylated form, may be produced by any suitable method known in the art. Such methods include constructing a nucleotide sequence encoding the polypeptide variant and expressing the sequence in a suitable transformed or transfected host. However, polypeptides for use in the invention may be produced, albeit less efficiently, by chemical synthesis or a combination of chemical synthesis or a combination of chemical synthesis and recombinant DNA technology.

The nucleotide sequence encoding an IFNB polypeptide for use in the present invention may be constructed by isolating or synthesizing a nucleotide sequence encoding the parent IFNB, e.g. with the amino acid sequence shown in SEQ ID NO:2, and then changing the nucleotide sequence so as to effect introduction (i.e. insertion or substitution) or removal (i.e. deletion or substitution) of the relevant amino acid residue(s).

The nucleotide sequence is conveniently modified by site-directed mutagenesis in accordance with well-known methods, see, e.g., Mark et al., “Site-specific Mutagenesis of the Human Fibroblast Interferon Gene”, Proc. Natl. Acad. Sci. USA, 81, pp. 5662-66 (1984); and U.S. Pat. No. 4,588,585.

Alternatively, the nucleotide sequence is prepared by chemical synthesis, e.g. by using an oligonucleotide synthesizer, wherein oligonucleotides are designed based on the amino acid sequence of the desired polypeptide, and preferably selecting those codons that are favoured in the host cell in which the recombinant polypeptide will be produced. For example, several small oligonucleotides coding for portions of the desired polypeptide may be synthesized and assembled by PCR, ligation or ligation chain reaction (LCR). The individual oligonucleotides typically contain 5′ or 3′ overhangs for complementary assembly.

Once assembled (by synthesis, site-directed mutagenesis or another method), the nucleotide sequence encoding the IFNB polypeptide is inserted into a recombinant vector and operably linked to control sequences necessary for expression of the IFNB variant in the desired transformed host cell.

A detailed description of the production of the IFNB variants disclosed herein, including suitable expression vectors, control sequences, host cells, production media, purification techniques, etc. can be found in the section entitled “Methods of preparing an interferon β polypeptide for use in the invention”, pp. 43-51, in WO 01/15736

The biological activity of the IFNB polypeptides can be assayed by any suitable method known in the art. Such assays include antibody neutralization of antiviral activity, induction of protein kinase, oligoadenylate 2,5-A synthetase or phosphodiesterase activities, as described in EP 0 41 313 B1. Such assays also include immunomodulatory assays (see, e.g., U.S. Pat. No. 4,753,795), growth inhibition assays, and measurement of binding to cells that express interferon receptors.

Specific assays for determining the biological activity of polypeptides or conjugates for use in the invention are disclosed in the Materials and Methods section herein.

Pharmaceutical Composition

The IFNB molecules can be used “as is” and/or in a salt form thereof. Suitable salts include, but are not limited to, salts with alkali metals or alkaline earth metals, such as sodium, potassium, lithium, calcium and magnesium, as well as e.g. zinc salts. These salts or complexes may by present as a crystalline and/or amorphous structure.

The IFNB molecule is preferably administered in a composition further including a pharmaceutically acceptable carrier or excipient. “pharmaceutically acceptable” means a carrier or excipient that does not cause any untoward-effects in patients to whom it is administered. Such pharmaceutically acceptable carriers and excipients are well known in the art.

The IFNB molecule can be formulated into pharmaceutical compositions by well-known methods. Suitable formulations are described in U.S. Pat. No. 5,183,746, Remington's Pharmaceutical Sciences by E. W. Martin, 18^{th} edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]).

The IFNB molecule may be formulated into a pharmaceutical composition in a variety of forms, including liquid, gel, lyophilized, pulmonary dispersion, or any other suitable form, e.g. as a compressed solid. The preferred form will depend upon the particular indication being treated and will be apparent to one of skill in the art.

The pharmaceutical composition may be administered parenterally (e.g. intravenously, intramuscularly, intraperitoneally, or subcutaneously), orally, intracerebrally, intradermally, intranasally, intrapulmonary, by inhalation, or in any other acceptable manner, e.g. using PowderJect or ProLease technology. The preferred mode of administration will depend upon the particular indication being treated and will be apparent to one of skill in the art.

A detailed description of suitable pharmaceutical compositions is given in the section entitled “Pharmaceutical composition and uses of a conjugate of the invention”, pp. 52-61 in WO 01/15736.

In a preferred embodiment of the invention the pharmaceutical composition comprises a sulfoalkyl ether cyclodextrin derivative, such as Captisol® (available from Cydex, Overland Park, Kans. 66213, US). Details concerning pharmaceutical compositions comprising the IFNB variants disclosed herein and sulfoalkyl ether cyclodextrin derivatives can be found in the section entitled “The sulfoalkyl ether cyclodextrin derivative”, pp. 37-49, in WO 03/002152.

Therapeutic Use

The variants and conjugates disclosed herein are useful for the therapeutic or prophylactic treatment of human diseases of the central nervous system or peripheral nervous system which have primarily neurological or psychiatric symptoms, ophthalmic diseases, cardiovascular diseases, cardiopulmonary diseases, respiratory diseases, kidney, urinary and reproductive diseases, gastrointestinal diseases and endocrine and metabolic abnormalities. In particular, such conditions and diseases include inflammatory, e.g. neuroinflammatory processes, which adversely affect excitable tissues, such as excitable tissues in the central nervous system tissue, peripheral nervous system tissue, cardiac tissue or retinal tissue such as, for example, brain, heart, or retina/eye.

Therefore, the variants disclosed herein can be used to treat or prevent damage to excitable tissue resulting from inflammatory processes in a variety of conditions and circumstances. Non-limiting examples of such conditions and circumstances are provided in Table 1 below.

In the example of the protection of neuronal tissue pathologies treatable in accordance with the present invention, such pathologies include those which result from reduced oxygenation of neuronal tissues. Any condition which reduces the availability of oxygen to neuronal tissue, resulting in stress, damage, and finally, neuronal cell death, can be treated by the methods of the present invention.

Generally referred to as hypoxia and/or ischemia, these conditions arise from or include, but are not limited to stroke, vascular occlusion, prenatal or postnatal oxygen deprivation, suffocation, choking, near drowning, carbon monoxide poisoning, smoke inhalation, trauma, including surgery and radiotherapy, asphyxia, epilepsy, hypoglycemia, chronic obstructive pulmonary disease, emphysema, adult respiratory distress syndrome, hypotensive shock, septic shock, anaphylactic shock, insulin shock, sickle cell crisis, cardiac arrest, dysrhythmia, nitrogen narcosis, and neurological deficits caused by heart-lung bypass procedures.

In one embodiment, the IFNB variants disclosed herein can be administered to prevent injury or tissue damage resulting from risk of injury or tissue damage during surgical procedures, such as, for example, tumor resection or aneurysm repair. Other pathologies caused by or resulting from hypoglycemia which are treatable by the methods described herein include insulin overdose, also referred to as iatrogenic hyperinsulinemia, insulinoma, growth hormone deficiency, hypocortisolism, drug overdose, and certain tumors.

Other pathologies resulting from excitable neuronal tissue damage include seizure disorders, such as epilepsy, convulsions, or chronic seizure disorders. Other treatable conditions and diseases include diseases such as stroke, hypotension, cardiac arrest, Alzheimer's disease, Parkinson's disease, cerebral palsy, brain or spinal cord trauma, AIDS dementia, age-related loss of cognitive function, memory loss, amyotrophic lateral sclerosis, seizure disorders, alcoholism, retinal ischemia, optic nerve damage resulting from glaucoma, and neuronal loss.

The IFNB variants disclosed herein may be used to treat conditions of, and damage to, retinal tissue. Such disorders include, but are not limited to retinal ischemia, macular degeneration, retinal detachment, retinitis pigmentosa, arteriosclerotic retinopathy, hypertensive retinopathy, retinal artery blockage, retinal vein blockage, hypotension, and diabetic retinopathy.

In another embodiment, the methods principles of the invention may be used to protect or treat injury resulting from radiation damage to excitable tissue. A further utility of the methods of the present invention is in the treatment of neurotoxin poisoning, such as domoic acid shellfish poisoning, neurolathyrism, and Guam disease, amyotrophic lateral sclerosis, and Parkinson's disease.

As mentioned above, the present invention is also directed to a method for enhancing excitable tissue function in a primate by peripheral administration of an Interferon-beta variant as described above. Various diseases and conditions are amenable to treatment using this method, and further, this method is useful for enhancing cognitive function in the absence of any condition or disease. These uses of the present invention are described in further detail below and include enhancement of learning and training in both human and non-human primates.

Conditions and diseases treatable by the methods of this aspect of the present invention directed to the central nervous system include but are not limited to mood disorders, anxiety disorders, depression, autism, attention deficit hyperactivity disorder, and cognitive dysfunction. These conditions benefit from enhancement of neuronal function. Other disorders treatable in accordance with the teachings of the present invention include sleep disruption, for example, sleep apnea and travel-related disorders; subarachnoid and aneurismal bleeds, hypotensive shock, concussive injury, septic shock, anaphylactic shock, and sequelae of various encephalitides and meningitides, for example, connective tissue disease-related cerebritides such as lupus. Other uses include prevention of or protection from poisoning by neurotoxins, such as domoic acid shellfish poisoning, neurolathyrism, and Guam disease, amyotrophic lateral sclerosis, Parkinson's disease; postoperative treatment for embolic or ischemic injury; whole brain irradiation; sickle cell crisis; and eclampsia.

Various neuropsychologic disorders, which are believed to originate from excitable tissue damage, are treatable by the methods disclosed herein. Chronic disorders in which inflammatory processes and hence neuronal damage is involved and for which treatment by the present invention is provided include disorders relating to the central nervous system and/or peripheral nervous system including age-related loss of cognitive function and senile dementia, chronic seizure disorders, Alzheimer's disease, Parkinson's disease, dementia, memory loss, amyotrophic lateral sclerosis, multiple sclerosis, tuberous sclerosis, Wilson's Disease cerebral and progressive supranuclear palsy, Guam disease, Lewy body dementia, prion diseases, such as spongiform encephalopathies, e.g., Creutzfeldt-Jakob disease, Huntington's disease, myotonic dystrophy, Freidrich's ataxia and other ataxias, as well as Gilles de la Tourette's syndrome, seizure disorders such as epilepsy and chronic seizure disorder, stroke, brain or spinal cord trauma, AIDS dementia, alcoholism, autism, retinal ischemia, glaucoma, autonomic function disorders such as hypertension and sleep disorders, and neuropsychiatric disorders that include, but are not limited to schizophrenia, schizoaffective disorder, attention deficit disorder, dysthymic disorder, major depressive disorder, mania, obsessive-compulsive disorder, psychoactive substance use disorders, anxiety, panic disorder, as well as unipolar and bipolar affective disorders. Additional neuropsychiatric and neurodegenerative disorders include, for example, those listed in the American Psychiatric Association's Diagnostic and Statistical manual of Mental Disorders (DSM), the most current version of which in incorporated herein by reference in its entirety.

The following table lists additional exemplary, non-limiting indications as to the various conditions and diseases amenable to treatment by the aforementioned Interferon-beta variants.

TABLE 1

| Cell, tissue or | Dysfunction or | | |
|---|---|---|---|
| organ | pathology | Condition or disease | Type |
| Heart | Ischemia | Coronary artery | Acute, chronic |
| | | disease | Stable, unstable |
| | | Myocardial | Dressler's syndrome |
| | | infarction | |
| | | Angina | |
| | | Congenital heart | Valvular |
| | | disease | Cardiomyopathy |
| | | Prinzmetal angina | |
| | | Cardiac rupture | Aneurysmatic |
| | | | Septal perforation |
| | | Angiitis | |
| | Arrhythmia | Tachy-, | Stable, unstable |
| | | bradyarrhythmia | Hypersensitive carotid |
| | | Supraventricular, | sinus node |
| | | ventricular | |
| | | Conduction | |
| | | abnormalities | |
| | Congestive heart | Left, right, biventricular | Cardiomyopathies, such |
| | failure | | as idiopathic familial, |
| | | | infective, metabolic, |
| | | | storage disease, |
| | | | deficiencies, connective |
| | | | tissue disorder, |
| | | | infiltration and |
| | | | granulomas, |
| | | | neurovascular |
| | | Myocarditis | Autoimmune, infective, |
| | | | idiopathic |
| | | Cor pulmonale | |
| | Blunt and penetrating | | |
| | trauma | | |
| | Toxins | Cocaine | |
| Vascular | Hypertension | Primary, secondary | |
| | Decompression | | |
| | sickness | | |
| | Fibromuscular | | |
| | hyperplasia | | |
| | Aneurysm | Dissecting, ruptured, | |
| | | enlarging | |
| Lungs | Obstructive | Asthma | |
| | | Chronic bronchitis, | |
| | | Emphysema and | |
| | | airway obstruction | |
| | Ischemic lung disease | Pulmonary | |
| | | embolism, | |
| | | Pulmonary | |
| | | thrombosis, | |
| | | Fat embolism | |
| | Environmental lung | | |
| | diseases | | |
| | Ischemic lung disease | Pulmonary embolism | |
| | | Pulmonary | |
| | | thrombosis | |
| | Interstitial lung | Idiopathic pulmonary | |
| | disease | fibrosis | |
| | Congenital | Cystic fibrosis | |
| | Cor pulmonale | | |
| | Trauma | | |
| | Pneumonia and | Infectious, parasitic, | |
| | pneumonitides | toxic, traumatic, | |
| | | burn, aspiration | |
| | Sarcoidosis | | |
| Pancreas | Endocrine | Diabetes mellitus, | Beta cell failure, |
| | | type I and II | dysfunction |
| | | | Diabetic neuropathy |
| | | Other endocrine cell | |
| | | failure of the | |
| | | pancreas | |
| | Exocrine | Exocrine pancreas | pancreatitis |
| | | failure | |
| Bone | Osteopenia | Primary | Hypogonadism |
| | | secondary | immobilisation |
| | | | Postmenopausal |
| | | | Age-related |
| | | | Hyperparathyroidism |
| | | | Hyperthyroidism |
| | | | Calcium, magnesium, |
| | | | phosphorus and/or |
| | | | vitamin D deficiency |
| | Osteomyelitis | | |
| | Avascular necrosis | | |
| | Trauma | | |
| | Paget's disease | | |
| Skin | Alopecia | Areata | Primary |
| | | Totalis | Secondary |
| | | | Male pattern baldness |
| | Vitiligo | Localized | Primary |
| | | generalized | secondary |
| | Diabetic ulceration | | |
| | Peripheral vascular | | |
| | disease | | |
| | Burn injuries | | |
| Autoimmune | Lupus | | |
| disorders | erythematodes, | | |
| | Sjiogren, | | |
| | Rheumatoid arthritis, | | |
| | Glomerulonephritis, | | |
| | Angiitis | | |
| | Langerhan's | | |
| | histiocytosis | | |
| Eye | Optic neuritis | | |
| | Blunt and penetrating | | |
| | injuries, Infections, | | |
| | Sarcoid, Sickle C | | |
| | disease, Retinal | | |
| | detachment, | | |
| | Temporal arteritis | | |
| Embryonic | Asphyxia | | |
| and fetal | | | |
| disorders | | | |
| | Ischemia | | |
| CNS | Chronic fatigue | | |
| | syndrome, acute and | | |
| | chronic hypoosmolar | | |
| | and hyperosmolar | | |
| | syndromes, AIDS | | |
| | Dementia, | | |
| | Electrocution | | |
| | Encephalitis | Rabies, Herpes | |
| | Meningitis | | |
| | Subdural hematoma | | |
| | Nicotine addiction | | |
| | Drug abuse and | Cocaine, heroin, | |
| | withdrawal | crack, marijuana, | |
| | | LSD, PCP, poly-drug | |
| | | abuse, ecstasy, | |
| | | opioids, sedative | |
| | | hypnotics, | |
| | | amphetamines, | |
| | | caffeine | |
| | Obsessive- | | |
| | compulsive disorders | | |
| | Spinal stenosis, | | |
| | Transverse myelitis, | | |
| | Guillian Barre, | | |
| | Trauma, Nerve root | | |
| | compression, | | |
| | Tumoral | | |
| | compression, Heat | | |
| | stroke | | |
| ENT | Tinnitus | | |
| | Meuniere's syndrome | | |
| | Hearing loss | | |
| | Traumatic injury, | | |
| | barotrauma | | |
| Kidney | Renal failure | Acute, chronic | Vascular/ischemic, |
| | | | interstitial disease, |
| | | | diabetic kidney disease, |
| | | | nephrotic syndromes, |
| | | | infections |
| | Henoch S. Purpura | | |
| Striated | Autoimmune | Myasthenia gravis | |
| muscle | disorders | Dermatomyositis | |
| | | Polymyositis | |
| | Myopathies | Inherited metabolic, | |
| | | endocrine and toxic | |
| | Heat stroke | | |
| | Crush injury | | |
| | Rhabdomylosis | | |
| | Mitochondrial | | |
| | disease | | |
| | Infection | Necrotizing fasciitis | |
| Sexual | Central and | Impotence secondary | |
| dysfunction | peripheral | to medication | |
| Liver | hepatitis | Viral, bacterial, | |
| | | parasitic | |
| | Ischemic disease | | |
| | Cirrhosis, fatty liver | | |
| | Infiltrative/metabolic | | |
| | diseases | | |
| Gastrointestinal | Ischemic bowel | | |
| | disease | | |
| | Inflammatory bowel | | |
| | disease | | |
| | Necrotizing | | |
| | enterocolitis | | |
| Organ | Treatment of donor | | |
| transplantation | and recipient | | |
| Reproductive | infertility | Vascular | |
| tract | | Autoimmune | |
| | | Uterine abnormalities | |
| | | Implantation | |
| | | disorders | |
| Endocrine | Glandular hyper- and | | |
| | hypofunction | | |

As mentioned above, these diseases, disorders or conditions are merely illustrative of the range of benefits provided by the IFNB variants disclosed herein. Accordingly, this invention generally provides therapeutic or prophylactic treatment of the consequences of mechanical trauma. Therapeutic or prophylactic treatment for diseases, disorders or conditions of the CNS and/or peripheral nervous system are preferred.

In a highly preferred embodiment of the invention the disease to be treated is stroke, such as ischemic or hemorrhagic stroke. In an ischemic stroke, the blood supply to part of the brain is cut off because either atherosclerosis or a blood clot has blocked a blood vessel. In an hemorrhagic stroke, a blood vessel bursts, preventing normal flow and allowing blood to leak into an area of the brain and destroy it.

With an ischemic stroke, blockage can occur anywhere along the arterial pathways to the brain. For example, a large deposit of fatty material (atheroma) can develop in a carotid artery, reducing its blood flow to a trickle. This condition is serious since each artery normally supplies a large percentage of the brain's blood supply. Fatty material may also break off from the wall of the carotid artery, travel with the blood, and become stuck in a smaller artery, blocking it completely. The carotid and vertebral arteries and their branches may become blocked in other ways. For example, a blood clot formed in the heart or on one of its valves can break loose (becoming an embolus), travel up through the arteries to the brain, and lodge there. The result is an embolic stroke. Such strokes are most common in people who have recently had heart surgery and in people who have defective heart valves or abnormal rythms (especially atrial fibrillation).

Most strokes begin suddenly, develop rapidly, and cause brain damage within minutes. Less commonly, strokes continue to worsen for several hours to a day or two as steadily enlarging are of the brain dies.

Symptoms that indicate a possible stroke require immediate medical attention; doctors can sometimes reduce the damage or prevent further damage by acting quickly. Many effects of a stroke require medical care, especially during the first few hours. At first, doctors usually administer oxygen and insert and intravenous line to make sure the patient receives fluids and nourishment. For a stroke in evolution, anticoagulants such as heparin may be given.

The efficiency of the IFNB variants described herein in treatment of stroke and related diseases may be assessed using various animal models known to the person skilled in the art. Numerous tests can be employed for the determination of whether the variants described herein have a beneficial effect in stroke and related diseases (mainly cerebral ischemia and spinal cord ischemia). Reference is made to the following relevant tests, but other tests may also prove suitable.
i) thromboembolic stroke model (cf Lapchak et al. Stroke 2002; 33:1665-1670 or Lapchak et al. Stroke 2002; 33:1411-1415),
ii) photothrombosis (cf. Zhao et al. Stroke 2002; 32:2157-2163),
iii) sub-arachnoid haemorrhage (cf Grasso et al. J. Neurosurgery 2002; 96:565-570),
iv) transient middle cerebral artery occlusion by aneurysm clips (cf Yenari et al. Neurological Research 2001; 23:72-78),
v) transient spinal cord ischemia (aneurysm clip, ballon) (cf Murakawi et al. Crit. Care Med. 2001; 29:814-818; Lips et al. Anesthesiol. 2000; 93:1303-1311; Lips et al. J. Neurosurg. Anesthesiol. 2002; 14:35-42; Lapchak et al. Stroke 2001; 33:1220-1225 or Sukarai et al. Stroke 2000; 31:200-207), and
   Materials and Methods
   Materials
   HeLa cells—(available from American Type Culture Collection (ATCC)
   ISRE-Luc (Stratagene, La Jolla USA)
   pCDNA 3.1/hygro (Invitrogen, Carlsbad USA)
   pGL3 basic vector (Promega)
   Human genomic DNA (CloneTech, USA)
   DMEM medium: Dulbecco's Modified Eagle Media (DMEM), 10% fetal bovine serum (available from Life Technologies A/S, Copenhagen, Denmark)
   Assays
   Interferon Assay Outline

It has previously been published that IFNB interacts with and activates Interferon type I receptors on HeLa cells. Consequently, transcription is activated at promoters containing an Interferon Stimulated Response Element (ISRE). It is thus possible to screen for agonists of interferon receptors by use of an ISRE coupled luciferase reporter gene (ISRE-luc) placed in HeLa cells.

Primary Assay

HeLa cells are co-transfected with ISRE-Luc and pCDNA 3.1/hygro and foci (cell clones) are created by selection in DMEM media containing Hygromycin B. Cell clones are screened for luciferase activity in the presence or absence of IFNB. Those clones showing the highest ratio of stimulated to unstimulated luciferase activity are used in further assays.

To screen muteins, 15,000 cells/well are seeded in 96 well culture plates and incubated overnight in DMEM media. The next day muteins as well as a known standard are added to the cells in various concentrations. The plates are incubated for 6 hours at 37° C. in a 5% CO₂ air atmosphere. LucLite substrate (Packard Bioscience, Groningen The Netherlands) is subsequently added to each well. Plates are sealed and luminescence measured on a TopCount luminometer (Packard) in SPC (single photon counting) mode. Each individual plate contains wells incubated with IFNB as a stimulated control and other wells containing normal media as an unstimulated control. The ratio between stimulated and unstimulated luciferase activity serves as an internal standard for both mutein activity and experiment-to-experiment variation.

Secondary Assay

Currently, there are 18 non-allelic interferon a genes and one IFNB gene. These proteins exhibit overlapping activities and thus it is critical to ensure that muteins retain the selectivity and specificity of IFNB.

The β-R1 gene is activated by IFNB but not by other interferons. The transciption of β-R1 thus serves as a second marker of IFNB activation and is used to ensure that muteins retain IFNB activity. A 300 bp promoter fragment of β-R1 shown to drive interferon sensitive transcription (Rani. M. R. et al (1996) JBC 271 22878-22884) was isolated by PCR from human genomic DNA and inserted into the pGL3 basic vector (Promega). The resulting β-R1:luciferase gene is used in assays similar to the primary assay described above. In astrocytoma cells, the resulting β-R1:luciferase gene has been described to show 250 fold higher sensitivity to IFNB than to interferon α (Rani et al. op cit).

ELISA Assay

The concentration of IFNB is quantitated by use of a commercial sandwich immunoassay (PBL Biomedical Laboratories, New Brunswick, N.J., USA). The kit is based on an ELISA with monoclonal mouse anti-IFNB antibodies for catching and detection of IFNB in test samples. The detecting antibody is conjugated to biotin.

Tests samples and recombinant human IFNB standard are added in 0.1 mL in concentrations from 10-0.25 ng/mL to microtiter plates, precoated with catching antibody. The plates are incubated at RT for 1 hr. Samples and standard are diluted in kit dilution buffer.

The plates are washed in the kit buffer and incubated with the biotinylated detecting antibody in 0.1 mL for 1 hr at RT. After another wash the streptavidin-horseradishperoxidase conjugate is added in 0.1 mL and incubated for 1 hr at RT.

The reaction is visualised by addition of 0.1 mL Tetramethylbenzidine (TMB) substrate chromogen. The plates are incubated for 15 minutes in the dark at RT and the reaction is stopped by addition of stop solution. The absorbanse is read at 450 nm using an ELISA reader.

Receptor Binding Assay

The receptor binding capability of a polypeptide or conjugate for use in the invention can be determined using the assay described in WO 95/25170 entitled “Analysis Of IFN-β(Phe₁₀₁) For Receptor Binding” (which is based on Daudi or A549 cells). Soluble domains of IFNAR1 and IFNAR2 can be obtained essentially as described by Arduini et al, Protein Science, 1999, vol. 8, 1867-1877 or as described in Example 9 herein.

Alternatively, the receptor binding capability is determined using a crosslinking agent such as disuccinimidyl suberate (DSS) available from Pierce, Rockford, Ill., USA as follows:

The polypeptide or conjugate is incubated with soluble IFNAR-2 receptor in the presence or absence of DSS in accordance with the manufacturer's instructions. Samples are separated by SDS-PAGE, and a western blot using anti-IFNB or anti-IFNAR2 antibodies is performed. The presence of a functional IFNB polypeptide/conjugate: receptor interaction is apparent by an increase in the molecular size of receptor and IFNB in the presence of DSS.

Furthermore, a crosslinking assay using a polypeptide or conjugate for use in the invention and both receptor subunits (IFNAR-1 and IFNAR-2) can establish Interferon receptor 1 binding ability. In this connection it has been published that IFNAR-1 binds only after an interferon β: IFNAR-2 complex is formed (Mogensen et al., Journal of Interferon and Cytokine Research, 19:1069-1098, 1999).

In Vitro Immunogenicity Tests

Reduced immunogenicity of a conjugate or polypeptide for use in the invention is determined by use of an ELISA method measuring the immunoreactivity of the conjugate or polypeptide relative to a reference molecule or preparation. The reference molecule or preparation is normally a recombinant IFNB preparation such as Avonex®, Rebif® or Betaseron®, or another recombinant IFNB preparation produced by a method equivalent to the way these products are made. The ELISA method is based on antibodies from patients treated with one of these recombinant IFNB preparations. The immunogenicity is considered to be reduced when the conjugate or polypeptide of the invention has a statistically significant lower response in the assay than the reference molecule or preparation.

Another method of determining immunogenicity is by use of sera from patients treated with IFNB (i.e. any commercial IFNB product) in an analogous manner to that described by Ross et al. J. Clin Invest. 95, 1974-78, 1995. In the antiviral neutralisation bioassay reduced immunogenicity results in reduced inhibition of a conjugate for use in the invention by patient sera compared to a wt IFNB reference molecule. Furthermore, in the biochemical IFN binding assay a less immunogenic conjugate is expected to bind to patient IgG to a lesser extent than reference IFNB molecules.

For the neutralisation assay, the reference and variant molecules are added in a concentration that produces approximately 80% virus protection in the antiviral neutralisation bioassay. The IFNB proteins are mixed with patient sera in various dilutions (starting at 1:20).

Antiviral Activity

The antiviral bioassay is performed using A549 cells (CCL 185, American tissue culture collection) and Encephalomyocarditis (EMC) virus (VR-129B, American tissue culture collection).

The cells are seeded in 96 well tissue culture plates at a concentration of 10,000 cells/well and incubated at 37° C. in a 5% CO₂ air atmosphere. A polypeptide or conjugate for use in the invention is added in concentrations from 100-0.0001 IU/mL in a total of 100 μl DMEM medium containing fetal calf serum and antibiotics.

After 24 hours the medium is removed and 0.1 mL fresh medium containing EMC virus is added to each well. The EMC virus is added in a concentration that causes 100% cell death in IFNB-free cell cultures after 24 hours.

After another 24 hrs, the antiviral effect of the polypeptide or conjugate is measured using the WST-1 assay. 0.01 mL WST-1 (WST-1 cell proliferation agent, Roche Diagnostics GmbH, Mannheim, Germany) is added to 0.1 mL culture and incubated for ½-2 hours at 37° C. in a 5% CO₂ air atmosphere The cleavage of the tetrazolium salt WST-1 by mitochondrial dehydrogenases in viable cells results in the formation of formazan that is quantified by measuring the absorbance at 450 nm.

Neutralisation of Activity in Interferon Stimulated Response Element (ISRE) Assay

The IFNB neutralising effect of anti-IFNB sera are analysed using the ISRE-Luciferase activity assay.

Sera from IFNB treated patients or from immunised animals are used. Sera are added either in a fixed concentration (dilution 1:20-1:500 (pt sera) or 20-600 ng/mL (animal sera)) or in five-fold serial dilutions of sera starting at 1/20 (pt sera) or 600 ng/mL (animal sera). IFNB is added either in five fold-dilutions starting at 25.000 IU/mL or in a fixed concentration (0.1-10 IU/mL) in a total volume of 80 μl DMEM medium+10% FCS. The sera are incubated for 1 hr. at 37° C. with IFNB.

The samples are then transferred to 96 well tissue culture plates containing HeLa cells transfected with ISRE-Luc grown from 24 hrs before (15,000 cells/well) in DMEM media. The cultures are incubated for 6 hours at 37° C. in a 5% CO₂ air atmosphere. LucLite substrate (Packard Bioscience, Groningen, The Netherlands) is subsequently added to each well. Plates are sealed and luminescence measured on a TopCount luminometer (Packard) in SPC (single photon counting) mode.

When IFNB samples are titrated in the presence of a fixed amount of serum, the neutralising effect was defined as fold inhibition (FI) quantified as EC50(w. serum)/EC50 (w/o serum). The reduction of antibody neutralisation of IFNB variant proteins is defined as

$$( 1 - \frac{FI variant}{FI wt} ) ⨯ 100 \%$$

Biological Half-Life Measurement

Measurement of biological half-life can be carried out in a number of ways described in the literature. One method is described by Munafo et al. European Journal of Neurology, 1998, vol 5 No 2 p 187-193, who used an ELISA method to detect serum levels of IFNB after subcutaneous and intramuscular administration of IFNB.

The rapid decrease of IFNB serum concentrations after i.v. administration has made it important to evaluate biological responses to IFNB treatment. However it is contemplated that the variants or conjugates for use in the present invention will have prolonged serum half-lifes also after i.v. administration making it possible to measure by e.g. an ELISA method or by the primary screening assay.

Different pharmacodynamic markers (e.g. serum neupterin and beta2 microglobulin) have also been studied (Clin Drug Invest (1999) 18(1):27-34). These can equally well be used to evaluate prolonged biological effect. These experiments may also be carried out in suitable animal species, e.g. rats.

Assays to assess the biological effects of IFNB such as antiviral, antiproliferative and immunomodulatory effects (as described in e.g. Annals of Neurology 1995 vol 37 No 1 p 7-15) can be used together with the primary and secondary screening assays described herein to evaluate the biological efficacy of the conjugate in comparison to wild type IFNB.

Accessible Surface Area (ASA)

The computer program Access (B. Lee and F. M. Richards, J. Mol. Biol. 55: 379-400 (1971)) version 2 (Copyright (c) 1983 Yale University) are used to compute the accessible surface area (ASA) of the individual atoms in the structure. This method typically uses a probe-size of 1.4 Å and defines the Accessible Surface Area (ASA) as the area formed by the centre of the probe. Prior to this calculation all water molecules and all hydrogen atoms are removed from the coordinate set, as are other atoms not directly related to the protein. Alternative programs are available for computing ASA, e.g. the program WhatIf G. Vriend, J. Mol. Graph. (1990) 8, 52-56, electronically available at the WWW interface on http://swift.embl-heidelberg.de/servers2/ (R. Rodriguez et. al. CABIOS (1998)14, 523-528.) using the option Accessibility to calculate the accessible molecular surface.

Fractional ASA of Side Chain

The fractional ASA of the side chain atoms is computed by division of the sum of the ASA of the atoms in the side chain with a value representing the ASA of the side chain atoms of that residue type in an extended ALA-x-ALA tripeptide. See Hubbard, Campbell & Thornton (1991) J. Mol. Biol. 220,507-530. For this example the CA atom is regarded as a part of the side chain of Glycine residues but not for the remaining residues. The following table indicates the 100% ASA standard for the side chain:

| | |
|---|---|
| Ala | 69.23 Å² |
| Arg | 200.35 Å² |
| Asn | 106.25 Å² |
| Asp | 102.06 Å² |
| Cys | 96.69 Å² |
| Gln | 140.58 Å² |
| Glu | 134.61 Å² |
| Gly | 32.28 Å² |
| His | 147.00 Å² |
| Ile | 137.91 Å² |
| Leu | 140.76 Å² |
| Lys | 162.50 Å² |
| Met | 156.08 Å² |
| Phe | 163.90 Å² |
| Pro | 119.65 Å² |
| Ser | 78.16 Å² |
| Thr | 101.67 Å² |
| Trp | 210.89 Å² |
| Tyr | 176.61 Å² |
| Val | 114.14 Å² |

Determining Surface Exposed Amino Acid Residues

The three-dimensional crystal structure of human interferon beta at 2.2 Å resolution (Karpusas et al. Proc. Nat. Acad. Sci. USA (1997) 94:11813-11818 is available from the Protein Data Bank (PDB) (Bernstein et. al. J. Mol. Biol. (1977) 112 pp. 535) and electronically available via The Research Collaboratory for Structural Bioinformatics PDB at http://www.pdb.org/ under accession code 1AU1. This crystal structure contain two independent molecules of human interferon beta in this example the A molecule is used.

Surface Exposure:

Using the WhatIf program as described above the following residues were found to have zero surface accessibility for their side chain atoms (for Gly the accessibility of the CA atom is used): G7, N14, C17, L21, I44, A55, A56, T58, I59, M62, L63, L98, L122, Y125, I129, L133, A142, W143, V146, I150, N153, I157, L160, T161, and L164.

Fractional Surface Exposure

For further analysis it was necessary to remodel the side chains of residues R71, R113, K115, L116, M117 due to steric clashes. The remodelling was done using Modeler 98, MSI INC. Performing fractional ASA calculations using the Access computer program on the remodelled interferon beta molecule (only including the amino acid residues and excluding the N-linked sugar moiety) resulted in the following residues having more than 25% of their side chain exposed to the surface: S2, N4, L5, F8, L9, R11, S12, F15, Q16 Q18, K19, W22, Q23, G26, R27, L28, E29, Y30, L32, K33, R35, M36, N37, D39, E42, K45, Q46, L47, Q48, Q49, Q51, K52, Q64, A68, R71, Q72, D73, S75, S76, G78, N80, E81, T82, E85, N86, A89, Y92, H93, N96, H97, K99, T100, E103, E104, K105, E107, K108, E109, D110, F111, R113, G114, K115, L116, S119, L120, H121, K123, R124, G127, R128, L130, H131, K134, A135, K136, E137, Y138, S139, H140, V148, R152, Y155, N158, G162, Y163, R165, and N166. and the following residues have more than 50% of their side chain exposed to the surface: N4, L5, F8, S12, F15, Q16, K19, W22, G26, R27, E29, Y30, K33, R35, N37, D39, E42, Q46, Q48, Q49, Q51, K52, R71, D73, S75, G78, N80, E81, T82, E85, N86, A89, Y92, H93, K99, T100, E103, E104, E107, K108, D110, F111, L116, K123, R124,G127,H131,K134, E137,V148,Y155, R165, and N166.

### EXAMPLES

### Example 1

Design of an Expression Cassette for Expression of IFNB in Mammalian and Insect Cells

The DNA sequence, GenBank accession number M28622 (shown in SEQ ID NO:1), encompassing a full length cDNA encoding human IFNB with its native signal peptide, was modified in order to facilitate high expression in mammalian cells. First the ATG start codon context was modified according to the Kozak consensus sequence (Kozak, M. J Mol Biol 1987 Aug. 20; 196(4):947-50), such that there is a perfect match to the consensus sequence upstream of the ATG start codon. Secondly the codons of the native human IFNB was modified by making a bias in the codon usage towards the codons frequently used in highly expressed human genes. Subsequently, certain nucleotides in the sequence were substituted with others in order to introduce recognition sites for DNA restriction endonucleases (this allows for easier modification of the DNA sequence later). Primers were designed such that the gene could be synthesised:

CBProFpr1
5′GGCTAGCGTTTAAACTTAAGCTTCGCCACCATGACCAACAAGTGCCTG
CTCCAGATCGCCCTGCTCCTGT-3′,
CBProFpr2
5′ACAACCTGCTCGGCTTCCTGCAGAGGAGTTCGAACTTCCAGTGCCAGA
AGCTCCTGTGGCAGCTGAACGG-3′,
CBProFpr3
5′GAACTTCGACATCCCCGAGGAAATCAAGCAGCTGCAGCAGTTCCAGAA
GGAGGACGCCGCTCTGACCATC-3′,
CBProFpr4
5′TTCCGCCAGGACTCCAGCTCCACCGGTTGGAACGAGACCATCGTGGAG
AACCTGCTGGCCAACGTGTACC-3′,
CBProFpr5
5′AGGAGAAGCTGGAGAAGGAGGACTTCACCCGCGGCAAGCTGATGAGCT
CCCTGCACCTGAAGCGCTACTA-3′,
CBProFpr6
5′GGAGTACAGCACTGCGCCTGGACCATCGTACGCGTGGAGATCCTGCGC
AACTTCTACTTCATCAACCGC-3′,
CBProFpr9
5′CACCACACTGGACTAGTGGATCCTTATCAGTTGCGCAGGTAGCCGGTC
AGGCGGTTGATGAAGTAGAAGT-3′,
CBProFpr10
5′AGGCGCAGTGGCTGTACTCCTTGGCCTTCAGGTAGTGCAGGATGCGGC
CATAGTAGCGCTTCAGGTGCAG-3′,
CBProFpr11
5′CTCCTTCTCCAGCTTCTCCTCCAGCACGGTCTTCAGGTGGTTGATCTG
GTGGTACACGTTGGCCAGCAGG-3′,
CBProFpr12
5′GAGCTGGAGTCCTGGCGGAAGATGGCGAAGATGTTCTGCAGCATCTCG
TAGATGGTCAGAGCGGCGTCCT-3′,
CBProFpr13
5′CCTCGGGGATGTCGAAGTTCATCCTGTCCTTCAGGCAGTACTCCAGGC
GCCCGTTCAGCTGCCACAGGAG-3′,
CBProFpr14
5′CAGGAAGCCGAGCAGGTTGTAGCTCATCGATAGGGCCGTGGTGCTGAA
GCACAGGAGCAGGGCGATCTGG-3′,

The primers were assembled to the synthetic gene by one step PCR using Platinum Pfx-polymerase kit (Life Technologies) and standard three step PCR cycling parameters. The assembled gene was amplified by PCR using the same conditions.

A cDNA encoding a N-terminal extended form of human IFNB was synthesised using the same PCR conditions as described above but with the primers CBProFpr1 and −14 substituted with the primers:

CBProFpr7
5′CTGCTCCAGATCGCCCTGCTCCTGTGCTTCAGCACCACGGCCCTATCG
ATGAAGCACCAGCACCAGCATC-3′,
CBProFpr8
5′CACTGCTTACTGGCTTATCGAAATTAATACGACTCACTATAGGGAGAC
CCAAGCTGGCTAGCGTTTAAAC-3′,
CBProFpr15
5′CAGGAAGCCGAGCAGGTTGTAGCTCATCTGTTGGTGTTGATGTTGGTG
CTGATGCTGGTGCTGGTGCTTC-3′,
CBProFpr16
5′AGCAGGGCGATCTGGAGCAGGCACTTGTTGGTCATGGTGGCGAAGCTT
AAGTTTAAACGCTAGCCAGCTT-3′,

in order to incorporate a purification TAG in the IFNB molecule.

The synthesised genes were cloned into pcDNA3.1/Hygro (Invitrogen) between the HindIII site at the 5′ end and the BamHI at the 3′, resulting in pCBProF1 and pCBProF2.

The synthetic intron from pCI-Neo (Promega) was amplified using standard PCR conditions as described above and the primers:

CBProFpr37
5′-CCGTCAGATCCTAGGCTAGCTTATTGCGGTAGTTTATCAC-3′,
CBProFpr38
5′-GAGCTCGGTACCAAGCTTTTAAGAGCTGTAAT-3′,

resulting in a 332 bp PCR fragment which was cut with NheI and HindIII and inserted in the 5′UTR of the plasmids pCBProF1 and pCBProF2 resulting in pCBProF4 and pCBProF5.

Codons for individual amino acids were changed by amplifying relevant regions of the coding region by PCR in such a way that the PCR introduced changes in the sequence can be introduced in the expression plasmids by classical cloning techniques. E.g. the primers:

Lys45arg-5′primer (NarI/KasI):
5′GCTGAACGGGCGCCTGGAGTACTGCCTGAAGGACAGGATGAACTTCGA
CATCCCCGAGGAAATCCGCCAGCTGCAGC-3′,
Lys45mut-3′primer (BsiWI):
5′TCTCCACGCGTACGATGGTCCAGGCGCAGTGGCTG-3′,

were used to introduce a K45R substitution in the PCR-fragment spanning the region from position 1055 to 1243 in pCBProF1. Both the PCR fragment and pCBProF1 was cut with NarI and BsiWI which are both unique. The PCR fragment and the vector backbone of pCBProF1 are purified and ligated resulting in substitution of the Lys45 codon AAG with the Arg codon CGC in pCBProF1.

Furthermore, SOE (sequence overhang extension) PCR was used for introduction of amino acid substitutions. In the SOE-PCR both the N-terminal part and the C-terminal part of the IFNB molecule were first amplified in individual primary PCRs.

For these primary PCRs the central complementary primers were synthesised such that the codon(s) for the amino acid(s) to be substituted is/are changed to the desired codon(s). The terminal primers were standard primers defining the N- and C-terminal of the IFNB molecule respectively. Further the terminal primers provided a restriction enzyme site enabling subsequent cloning of the full-length PCR product. Thus, the central (nonsense) primer and the N-terminal (sense) primer were used to amplify the N-terminal part of the IFNB coding region in one of the primary PCRs and equivalently for the C-terminal part. Once amplified the N- and C-terminal parts are assembled into the full-length product in a secondary PCR and cloned into a modified version of pcDNA3.1/Hygro as described above. For instance, the following primers were used to introduce the mutations for the substitutions F111N and R113T:

CBProFprimer9 (Sense):
CACCACACTGGACTAGTGGATCCTTATCAGTTGCGCAGGTAGCCGGTCAG
GCGGTTGATGAAGTAGAAGT,
CBProFprimer231 (Antisense):
CATCAGCTTGCCGGTGGTGTTGTCCTCCTTC,
CBProFprimer230 (Sense):
GAAGGAGGACAACACCACCGGCAAGCTGATG,
CBProFprimer42 (Antisense):
CACACTGGACTAGTAAGCTTTTATCAGTTGCGCAGGTAGC,

Furthermore, in cases where the introduced mutation(s) were sufficiently close to a unique restriction endo-nuclease site in the expression plasmid variant genes were constructed using construction procedure encompassing a single PCR step and a subsequent cloning. For instance, the substitution K19R was introduced by use of the PCR primer:

CBProFpr58:
GAGGAGTTCGAACTTCCAGTGCCAGCGCCTCCTGTGGCAGCTGAACG,

CBProF Primer9.

The PCR product was subsequently cloned using the restriction endo-nuclease sites BsiWI and BstBI.

### Example 2

Construction and Expression of an IFNB Variant with One Introduced Glycosylation Site in Position 111

In order to insert an extra N-linked glycosylation site at position 111 in human IFNB, the synthetic gene (hinf-β) encoding human IFNB (described in Example 1) was altered by site-directed PCR mutagenesis. Using BIO-X-ACT (Bioline, UK) and the plasmid PF050 [hinf-β)/pcDNA3.1(−)Hygro/Intron (a derivative of pcDNA3.1(−)Hygro (Invitrogen, USA) in which a chimeric intron obtained from pCI-neo (Promega, USA) had been inserted between the BamHI and NheI sites in the MCS of the vector] as template, two PCR reactions were performed with two overlapping primer-sets [CB41 (5′-TTTAAACTGGATCCAGCCACCATGACCAACAAG-3′) ICB55 (5′-CGGCCATAGT AGCGCTTCAGGTGCAGGGAGCTCATCAGCTTGCCGGTGGTGTTGTCCTCCTTC-3′) and CB42/CB86 (5′-GAAGGAGGACAACACCACCGGCAAGCTGATGAGCTCCCTGCACCTGAAGCGCTAC TATGGCC G-3′) resulting in two fragments of 446 and 184 base pairs, respectively. These two fragments were assembled in a third PCR with the flanking primers CB41 and CB42. The resulting gene was inserted into the mammalian expression vector pcDNA3.1 (−)Hygro/Intron and confirmed by DNA sequencing to have the correct base changes leading to the substitutions F111N and R113T in human IFNB (plasmid designated PF085).

To test the activity of the [F111N+R113T] human IFNB variant, PF085 was transfected into the CHO K1 cell line (ATCC #CCL-61) by use of Lipofectamine 2000 (Life Technologies, USA) as transfection agent. 24 hours later the culture medium was harvested and assayed for IFNB activity/concentration:

| | |
|---|---|
| Activity: | 56046 IU/ml [primary assay] |
| ELISA: | 80 ng/ml |
| Specific activity: | 7 × 10⁸ IU/mg |

As can be seen, the [F111N+R113T] human IFNB variant has a very high specific activity, about twice the specific activity of wild-type human IFNB.

### Example 3

Construction and Expression of an IFNB Variant with One Introduced Glycosylation Site in Position 49

Analogously to what is described in Example 5 of WO 01/15736 an extra N-linked glycosylation site was introduced in position 49 by means of the substitutions Q49N and Q51T. Using PF043 (hinf-β/pcDNA3.1 (Invitrogen, USA)) as template, two PCR reactions were performed with two overlapping primer-sets [PBR7]/PBR78 (5′-GGCGTCCTCCTTGGTGAAGTTCTGCAGCTG-3′) and PBR8 (5′-ATATATCCCAAGCTTTTATCAGTTGCGCAGGTAGCCGGT-3′) IPBR77 (5′-CAGCTGCAGAACTTCACCAAGGAGGACGCC-3′) resulting in two fragments of 228 and 369 base pairs, respectively. These two fragments were assembled in a third PCR with the flanking primers PBR7 and PBR8. The resulting gene was inserted into the mammalian expression vector pcDNA3.1(−)Hygro/Intron and confirmed by DNA sequencing to have the correct base changes leading to [Q49N, Q51T] human IFNB (plasmid designated PF104).

To test the activity of the [Q49N+Q51T] human IFNB variant, PF104 was transfected into the CHO K1 cell line by use of Lipofectamine 2000 (Life Technologies, USA) as transfection agent. 24 hours later the culture medium was harvested and assayed for IFNB activity/concentration:

| | |
|---|---|
| Activity: | 17639 IU/ml [primary assay] |
| ELISA: | 10 ng/ml |
| Specific activity: | 1.7 × 10⁹ IU/mg |

The [Q49N+Q51T] human IFNB variant has a high specific activity. This may be due to poor recognition by one of the monoclonal antibodies used in the ELISA.

### Example 4

Construction and Expression of an IFNB Variant with Two Introduced Glycosylation Sites

The additional glycosylation sites described in Examples 5 and 6 of WO 01/15736 were introduced into human IFNB by means of the substitutions Q49N, Q51T, F111N, and R113T.

Using PF085 (described in example 5 of WO 01/15736) as template, two PCR reactions were performed with two overlapping primer-sets [PBR89 (5′CGCGGATCCAGCCACCATGACCAACAAGTGCCTG)/PBR78 and PBR8/PBR77] resulting in two fragments of 228 and 369 base pairs, respectively.

These two fragments were assembled in a third PCR with the flanking primers PBR89 and PBR8. The resulting gene was inserted into the mammalian expression vector pcDNA3.1(−)Hygro/Intron and confirmed by sequencing to have the correct base changes leading to [Q49N, Q51T, F111N, R113T] human IFNB (plasmid designated PF123).

PF123 was transfected into CHO K1 cells by use of Fugene 6 (Roche) as transfection agent. 24 hours later the culture medium was harvested and assayed for IFNB activity/concentration:

| | |
|---|---|
| Activity: | 29401 IU/ml [primary assay] |
| ELISA: | 14 ng/ml |
| Specific activity: | 2.1 × 10⁹ IU/ml |

Evidently, the [Q49N+Q51T+F111N+R113T] human IFNB variant also has a high specific activity.

The variant was found to have receptor binding activity in the receptor binding assay described in the Materials and Methods section, which is based on the use of the cross-linking agent DSS.

### Example 5

Production of the [Q49N, Q51T, F111N, R113T] human IFNB glycosylation variant in Roller Bottles

A CHOK1 sub-clone (5/G-10) producing the [Q49N+Q51T+F111N+R113T] glycosylation variant was seeded into 2 roller bottles, each with an expanded surface of 1700 cm² (Corning, USA), in 200 ml DMEM/F-12 medium (LifeTechnologies; Cat. # 31330) supplemented with 10% FBS and penicillin/streptomycin (P/S). After 2 days the medium was exchanged. After another 2 days the two roller bottles were nearly 100% confluent and the medium was shifted to 300 ml serum-free UltraCHO medium (BioWhittaker; Cat. # 12-724) supplemented with 1/500 EX-CYTE (Serologicals Proteins; Cat. # 81129N) and P/S. Growing the cells in this medium promotes a higher cell mass, higher than can be achieved in the serum containing medium. After 2 days the medium was renewed. After another 2 days the medium was shifted to the production medium: DMEM/F-12 medium (Life Technologies; Cat. # 21041) supplemented with 1/100 ITSA (Life Technologies; Cat. # 51300-044) [ITSA stands for Insulin (1.0 g/L)—Transferrin (0.55 g/L)—Selenium (0.67 mg/L) supplement for Adherent cultures], 1/500 EC-CYTE and P/S. The harvested media from the two roller bottles were pooled before a medium sample was taken out for IFNB activity determination.

### Example 6

Production, Purification, and PEGylation of [K19R, K45R, K123R] Human IFNB

To end up with 100 ml serum-free medium containing the IFNB variant K19R+K45R+K123R, 3 T-175 flasks were seeded with COS-7 cells in DMEM medium (Life technologies; Cat. # 21969-035) supplemented with 10% FBS plus Glutamine and penicillin/streptomycin. On the day of transfection (at nearly 100% confluency) the medium was renewed with 30 ml fresh medium 45 hours before the transfection. To prepare the transfection, 1890 μl DMEM medium without supplements was aliquoted into a 14 ml polypropylene tube (Corning). 210 μl Fugene 6 (Roche) was added directly into the medium and incubated for 5 min at RT. In the meantime 168 μg plasmid DNA ([K19R, K45R, K123R]INF-β/pcDNA3.1(−)Hygro; PF #161) was aliquoted into another 14 ml polypropylene tube. After 5 min incubation the Fugene 6 mix was added directly to the DNA solution and incubated for 15 min at RT. After incubation about 700 μl was added drop wise to each of the three cell media.

Next day the transfection medium was substituted with 35 ml serum-free production medium. The serum-free medium is based on DMEM medium (Life Technologies; Cat. # 31053-028) supplemented with Glutamine, Sodium Pyruvate, penicillin/streptomycin, 1% ITSA (Life Technologies; Cat. # 51300-044), and 0.2% Ex-Cyte (Serologicals Proteins; Cat. # 81-129). Before the production medium was added the cell layers were washed two times in the DMEM medium without additives.

Three days post-transfection the 100 ml serum-free medium was harvested for purification and PEGylation of the IFNB variant.

pH was adjusted to 6.8 and conductivity adjusted to <10 mS/cm with Milli Q water. Then the broth was batch adsorbed to 1 ml SP 550 cation exchange resin (TosoHaas) preequilibrated with buffer A (20 mM phosphate, 100 mM NaCl, pH 7). After 2 h rotation end over end, the resin was allowed to sediment and transferred to a column. The resin was washed with 5 column volumes buffer A and eluted with 2 ml buffer B (20 mM phosphate, 800 mM NaCl, pH 7). The eluate was concentrated to 500 ul on VivaSpin (cutoff 10 kDa) after addition of 5% ethyleneglycol. The concentrate was adjusted to 50 mM phosphate, 0.3 M NaCl, 20% ethyleneglycol, pH 8 in a final volume of 2 ml and further concentrated to 0.5 ml.

The final concentrate was PEGylated as follows: to 100 ul of the final concentrate, 25 ul of activated mPEG-SPA (5000 kDa, Shearwater, Ala.) freshly prepared in phosphate buffer, pH 8 were added to make final concentrations of activated PEG of 0, 5, 10, 25 or 50 mg/ml. The reaction was allowed to proceed for 30 min at room temperature and then quenched by addition of 50 mM glycine buffer. Samples were frozen immediately at −80° C. and bioactivity was measured as described (Primary Assay). Western blots of each sample were performed in order to evaluate the amount of unreacted IFNB variant present in the PEGylated sample.

Results demonstrate that at 25 mg activated PEG/ml, nonPEGylated IFNB variant was absent as judged by western blot and the variant retained 50% of its bioactivity compared to the control sample (treated identically, but with 0 mg/ml activated PEG).

### Example 7

Variants Having Increased Carbohydrate Attachment at Position 49

The inserted N-linked glycosylation site at position 49 in the IFNB variant [Q49N, Q51T] described in Example 6 of WO 01/15736 is used only about 60%. In order to increase the amount of attached carbohydrate the glutamine residue at position 48 was exchanged with phenylalanine (Q48F), valine (Q48V), and tryptophan (Q48W) by site-directed PCR mutagenesis. Using BIO-X-ACT (Bioline, UK) and PF185 (PF185 contains the same cDNA sequence as PF104, described in example 6, despite the fact that a Kozak sequence has been inserted in front of the start ATG) as template, PCR reactions were performed with overlapping primer-sets:

Q48F, Q49N, Q51T
PBR89
(5′CGCGGATCCAGCCACCATGACCAACAAGTGCCTG)/
PBR148
(5′GTCCTCCTTGGTGAAGTTGAACAGCTGCTT)
and
PBR8
((5′-ATATATCCCAAGCTTTTATCAGTTGCGCAGGTAGCCGGT-3′))/
PBR147
(5′AAGCAGCTGTTCAACTTCACCAAGGAGGAC)
Q48V, Q49N, Q51T
PBR89
(5′CGCGGATCCAGCCACCATGACCAACAAGTGCCTG)/
PBR150
(5′GTCCTCCTTGGTGAAGTTCACCAGCTGCTT)
and
PBR8/PBR149
(5′AAGCAGCTGGTGAACTTCACCAAGGAGGAC)
Q48W, Q49N, Q51T
PBR89
(5′CGCGGATCCAGCCACCATGACCAACAAGTGCCTG)/
PBR152
(5′GTCCTCCTTGGTGAAGTTCCACAGCTGCTT)
and
PBR8/PBR151
(5′AAGCAGCTGTGGAACTTCACCAAG GAGGAC)

The fragments were assembled in PCR reactions with the flanking primers PBR89 and PBR8. The resulting genes were inserted into the mammalian expression vector pcDNA3.1 (−)Hygro/Intron and confirmed by sequencing to have the correct base changes leading to [Q48F, Q49N, Q51T] human IFNB (plasmid designated PF305), [Q48V, Q49N, Q51T] human IFNB (plasmid designated PF306), and [Q48W, Q49N, Q51T] human IFNB (plasmid designated PF307), respectively.

PF305, PF306, PF307, and PF185 (encoding [Q49N, Q51T] human IFNB) were ansfected into CHO K1 cells by use of Fugene 6 (Roche) as transfection agent. 24 hours later the culture medium was harvested and assayed for IFNB activity:

| | |
|---|---|
| PF185 | 134713 IU/ml |
| PF305 | 53122 IU/ml |
| PF306 | 65949 IU/ml |
| PF307 | 45076 IU/ml |

In order to evaluate the amount of attached carbohydrate in these glycosylation variants a Western blot was performed with equal amount of activity in each lane. As was seen the amino acid exchanges (Q48F, Q48V, Q48W) in front of the introduced glycosylation site (Q49N, Q51T) all leads to an increased amount of fully glycosylated material.

In another experiment it was seen that insertion of especially tyrosine in position 48 lead to an increased amount of attached carbohydrate to the inserted N-linked glycosylation site in position 49.

### Example 8

Variants Having Increased Carbohydrate Attachment at Position 111

The inserted N-linked glycosylation site at position 111 in the IFNB variant [F111N, R113T] described in Example 5 of WO 01/15736 is used only about 50%. In order to increase the amount of attached carbohydrate the aspartic acid residue at position 110 was exchanged with phenylalanine (D110F) and valine (D110V) by site-directed PCR mutagenesis. Using BIO-X-ACT (Bioline, UK) and PF085 (described in Example 5 of WO 01/15736) as template, PCR reactions were performed with overlapping primer-sets:

D110F, F111N, R113T
PBR89
(5′CGCGGATCCAGCCACCATGACCAACAAGTGCCTG)/
PBR154
(5′CAGCTTGCCGGTGGTGTTGAACTCCTTCTC)
and
PBR8/PBR153
(GAGAAGGAGTTCAACACCACCGGCAAG CTG)
D110V, F111N, R113T
PBR89
(5′CGCGGATCCAGCCACCATGACCAACAAGTGCCTG)/
PBR156
(5′CAGCTTGCCGGTGGTGTTCACCTCCTTCTC)
and
PBR8/PBR155
(5′GAGAAGGAGGTGAACACCACCGGCAAGCTG)

The fragments were assembled in PCR reactions with the flanking primers PBR89 and PBR8. The resulting genes were inserted into the mammalian expression vector pcDNA3.1 (−)Hygro/Intron and confirmed by sequencing to have the correct base changes leading to [D110F, F111N, R113T] human IFNB (plasmid designated PF308) and [D110V, F111N, R113T] human IFNB (plasmid designated PF309), respectively.

PF308, PF309 and PF085 (encoding [F111N, R113T] human IFNB) were transfected into CHO K1 cells by use of Fugene 6 (Roche) as transfection agent. 24 hours later the culture medium was harvested and assayed for IFNB activity:

| | |
|---|---|
| PF085 | 58615 IU/ml |
| PF308 | 50900 IU/ml |
| PF309 | 15063 IU/ml |

In order to evaluate the amount of attached carbohydrate in these glycosylation variants a Western blot was performed with equal amount of activity in each lane. As was seen the amino acid exchanges (D110F and D110V) in front of the introduced glycosylation site (F111N, R113T) both leads to a significantly increased amount of fully glycosylated material.

In another experiment it was seen that insertion of especially tyrosine in position 110 lead to an increased amount of attached carbohydrate to the inserted N-linked glycosylation site in position 111.

### Example 9

Separation of IFNB Polypeptide Glycoforms

Hydroxyapatite chromatography is an efficient means for separation of IFNB glycoforms and, e.g., obtain glycoforms with fully utilized glycosylation sites. This is illustrated in the present example.

The IFNB variant [Q49N+Q51T+F111N+R113T] produced as described in Example 8 of WO 01/15736 was purified in a three-step procedure:

The harvested media from roller bottles was centrifuged and filtered through a 0.22 um filter (PVDF). The filtrated media was diafiltrated on a Vivaflow 200 system equipped with a polyethersulfon membrane with cut off 10000 and applied to a S-Sepharose column (Pharmacia) equilibrated with 50 mM sodium acetate, 50 mM sodium chloride, pH 5.5. The IFNB variant bound to the column was eluted with 50 mM sodium acetate, 0.5 M sodium chloride, pH 5.5. The concentration of sodium chloride in the eluate from the S-Sepharose column was adjusted to 1.0 M and the sample was applied on a Phenyl-Sepharose High Performance column (Pharmacia) equilibrated with 50 mM sodium acetate, 1.0 M sodium chloride, pH 5.5. Following application the column was washed with Milli Q water. The IFNB variant was eluted with a gradient from Milli Q water to 60% ethylene glycol, 50 mM sodium acetate, pH 5.5 in 30 column volumes. Fractions containing fully glycosylated IFNB variant were collected and the buffer in the eluate was changed to 15 mM sodium phosphate buffer, pH 7.2. The sample was applied on a hydroxyapatite column (CHT II, Ceramic hydroxyapatite, Type II, Biorad) equilibrated with 15 mM sodium phosphate. The fully glycosylated form passed through the column whereas the underglycosylated form with one extra site used bound to the column and was eluted with a linear sodium phosphate gradient from 15 mM to 200 mM in 20 column volumes.

The purity of fully glycosylated [Q49N+Q511T+F111N+R113T] IFNB was judged to be higher than 95% based on SDS-PAGE.

### Example 10

PEGylation of IFNB Variants with Introduced Glycosylation Sites

A fresh stock solution of SCM-PEG (succinimidyl ester of carboxymethylated PEG from Shearwater, Ala., 5 kDa or 12 kDa) was prepared in methanol before each experiment.

100 microliter of a 0.3 mg/ml solution of the glycovariant [Q49N+Q51T+F111N+R113T] human IFNB in 50 mM sodium phosphate, 100 mM sodium chloride, pH 7.0 were PEGylated with SCM-PEG, 5 kDa or 12 kDa, with two times molar surplus of PEG to possible PEGylation sites, i.e. lysines and N-terminus. After incubation for 30 min at room temperature, the reaction was quenched by addition of 5 μl 20 mM glycine, pH 8.0. At this stage, the reaction mixture contained a minor part of unmodified protein judged by SDS-PAGE. In vitro testing using the primary screening assay demonstrated that the pegylated material retained 40% activity with 1-3 groups of 12 kDa PEG attached. With 1-3 groups of 5 kDa PEG attached the retained bioactivity was 25%.

In another experiment 50 μl of purified [Q49N+Q51T+F111N+R113T+K19R+K45R+K123R] human IFNB with a protein concentration of 0.1 mg/ml was PEGylated in 50 mM sodium phosphate, 100 mM sodium chloride, pH 8.0 with SCM-PEG, 5 kDa, with 20 times molar excess of PEG to possible PEGylation sites, i.e. lysines and N-terminus. After incubation for 30 min at room temperature, the reaction was quenched by addition of 5 μl 20 mM glycine, pH 8.0. At this stage, the reaction mixture contained a minor part of unmodified protein judged by SDS-PAGE.

In vitro testing using the primary screening assay demonstrated that the pegylated material retained 45% activity with 1-3 groups of 5 kDa PEG attached. A higher molar surplus of PEG was needed to PEGylate variants in which one or several lysines were substituted with other amino acid residues.

Pegylated material was separated from un-pegylated material and surplus of PEG using either size-exclusion chromatography or cation exchange chromatography or a combination of both. Size-exclusion chromatography was performed with a Superose 12 or Superdex 75 column from Pharmacia equilibrated with PBS buffer, pH 7.2. Cation exchange chromatography was performed on SP-Sepharose HP (Pharmacia) equilibrated with 20 mM citrate, pH 2.7. Elution from the SP-Sepharose HP column was performed either by increasing the concentration of salt (e.g. sodium chloride) or by increasing the pH of the buffer (e.g. sodium acetate or sodium phosphate).

### Example 11

Stabilization of Glycosylated IFNB Variant by Performing the Substitution of C17S

CHO-K1 cells were transfected with plasmids encoding two hyper-glycosylated IFNB variants: [S2N, N4T, Q51N, E53T] human IFNB (PF276) and [S2N, N4T, C17S, Q51N, E53T] human IFNB (PF279). Confluent stable primary transfection pools were expanded into four T-175 flasks each. At confluency, the flasks were shifted from serum containing medium to a serum-free medium based on DMEM/F-12 medium (Lifetecnologies #21045-025) supplemented with 1/100 ITSA (Life Technologies #51300-044) and 1/1000 Ex-Cyte (Serologicals Corp. #81-129). Every day, in 15 days, 120 ml of each variant was harvested and frozen at −80° C.

The supernatants from the daily harvest were collected and filtered through 0.22 um filter (PVDF based). The supernatant was concentrated approximately 15 times on a Vivaflow 200 system equipped with a polyethersulfon membrane with cut-off 10000 and the concentrated sample was applied on a S-Sepharose column equilibrated with 50 mM sodium acetate, 50 mM NaCl, pH 5.5 The IFNB variant eluted in a step with 50 mM sodium acetate, 0.5 M NaCl, pH 5.5.

The concentration of sodium chloride in the eluate from the S-Sepharose column was adjusted to 1.0 M and the sample was applied on a Phenyl-Sepharose column equilibrated with 50 mM sodium acetate, 1.0 M sodium chloride, pH 5.5. Extensive washing with the equilibration buffer was carried out before the IFNB variant was eluted with 60% ethylene glycol in 50 mM sodium acetate, pH 5.5.

Unreduced SDS-PAGE following the purification clearly demonstrated the formation of dimer with [S2N, N4T, Q51N, E53T] human IFNB, whereas no dimer was present with [S2N, N4T, C17S, Q51N, E53T] human IFNB.

### Example 12

Production, Purification and PEGylation of the [C17S+Q49N+Q51T+D110F+F111N+R113T] Human IFNB Variant

A CHOK1 sub-clone (5/G-10) producing [C17S+Q49N+Q51T+D110F+F111N+R113T] human IFNB glycosylation variant was seeded into 6 roller bottles, each with an expanded surface of 1700 μm² (Corning, USA), in 200 ml DMEM/F-12 medium (LifeTechnologies; Cat. # 31330) supplemented with 10% FBS and penicillin/streptomycin (P/S). After 2 days the medium was exchanged. After another 2 days the two roller bottles were nearly 100% confluent and the medium was shifted to 300 ml serum-free UltraCHO medium (BioWhittaker; Cat. # 12-724) supplemented with 1/500 EX-CYTE (Serologicals Proteins; Cat. # 81129N) and P/S. Growing the cells in this medium promotes a higher cell mass, higher than can be achieved in the serum containing medium. After 2 days the medium was renewed. After another 2 days the medium was shifted to the production medium: DMEM/F-12 medium (Life Technologies; Cat. # 21041) supplemented with 1/100 ITSA (Life Technologies; Cat. # 51300-044) [ITSA stands for Insulin (1.0 g/L)—Transferrin (0.55 g/L)—Selenium (0.67 mg/L) supplement for Adherent cultures], 1/500 EC-CYTE and P/S. The harvested media from the roller bottles were pooled before a medium sample was taken out for IFNB activity determination. Every day, in 21 days, 1.8 l medium was harvested and frozen at −80° C.

The harvested media from roller bottles was centrifuged and filtered through a 0.22 μm filter (PVDF). The filtrated media was diafiltrated on a Vivaflow 200 system equipped with a polyethersulfon membrane with cut off 10000 and applied to a S-Sepharose column (Pharmacia).

The S-Sepharose column was equilibrated with 50 mM sodium acetate, 50 mM sodium chloride, pH 5.5 and the interferon variant was eluted with 50 mM sodium acetate, 0.5 M sodium chloride, pH 5.5. The concentration of sodium chloride in the eluate was adjusted to 1.0 M.

The eluate from the S-Sepharose column was applied on a Phenyl-Sepharose High Performance column (Pharmacia) equilibrated with 50 mM sodium acetate, 1.0 M sodium chloride, pH 5.5. Following application the column was washed with 50 mM sodium acetate, 50 mM sodium chloride, pH 5.5. The IFNB variant was eluted with a gradient from 50 mM sodium acetate, 50 mM sodium chloride, pH 5.5 to 60% ethylene glycol, 50 mM sodium acetate, pH 5.5 in 30 column volumes. Fractions containing fully glycosylated IFNB variant were collected and pooled.

The ethylene glycol in the eluate from the Phenyl-Sepharose was removed by passing the eluate through a S-Sepharose column equilibrated with 50 mM sodium acetate, 50 mM sodium chloride, pH 5.5. The ethylene glycol was in the flow through where as the interferon variant bound to the column. Following application the column was washed with 20 mM sodium acetate, pH 5.5 and the interferon variant was eluted with 100 mM sodium phosphate, pH 7.5.

The phosphate concentration in the eluate was adjusted to 15 mM sodium phosphate buffer, pH 7.2. and applied on a hydroxyapatite column (CHT I, Ceramic hydroxyapatite, Type I, Biorad) equilibrated with 15 mM sodium phosphate, pH 7.2. The fully glycosylated form passed through the column where as the underglycosylated form with one extra site used bound to the column and was eluted with a linear sodium phosphate gradient from 15 mM to 200 mM sodium phosphate, pH 6.8 in 20 column volumes.

The purity of the fully glycosylated [C17S+Q49N+Q51T+D110F+F111N+R113T] human IFNB variant was judged to be higher than 95% based on SDS-PAGE.

Following purification the variant was PEGylated. A fresh stock solution of 10 mg/ml SCM-PEG (succinimidyl ester of carboxymethylated PEG from Shearwater, Ala., 12 K or 20 K) was prepared in 96% ethanol before each experiment.

A protein solution of 0.1 mg/ml in 20 mM sodium phosphate, pH 7.0 was PEGylated with SCM-PEG, 20K, with 0.75 times molar surplus of PEG to possible PEGylation sites, i.e. lysines and N-terminus. After incubation for 30 min at room temperature, the reaction was quenched by addition of a surplus of 20 mM glycine, pH 8.0. The reaction mixture contained a mixture of mono-, di- and un-pegylated material. Mono-pegylated material was separated from other species using either cation exchange chromatography or size-exclusion chromatography or a combination of both. pH in the PEGylation solution was adjusted to pH 2.7 and the sample was applied on a SP-Sepharose HR (Pharmacia) column equilibrated with 20 mM sodium citrate, pH 2.7. The pegylated protein was eluted from the column with 50 mM sodium acetate containing 1 M sodium chloride and applied on a size-exclusion column, Sephacryl S-100, ((16/60) Pharmacia) equilibrated with 100 mM sodium acetate, 200 mM sodium chloride, pH 5.5. Fractions containing mono-pegylated material were pooled and characterized further.

In another experiment a protein solution of 0.16 mg/ml in 20 mM sodium phosphate, pH 7.0 was PEGylated with SCM-PEG, 12K, with 2 times molar surplus of PEG to possible PEGylation sites, i.e. lysines and N-terminus. After incubation for 30 min at room termperature, the reaction was quenched by addition of a surplus of 20 mM glycine, pH 8.0. The reaction mixture contained a mixture of mono-, di-, tri-pegylated material together with underivatized material. The pegylated material was separated from the unmodified protein using either cation exchange chromatography or size-exclusion chromatography or a combination of both. pH in the PEGylation solution was adjusted to pH 2.7 and the sample was applied on a SP-Sepharose HR (Pharmacia) column equilibrated with 20 mM sodium citrate, pH 2.7. The pegylated protein was eluted from the column with 50 mM sodium acetate containing 1 M sodium chloride and applied on a size-exclusion column, Sephacryl S-100, ((16/60) Pharmacia) equilibrated with 100 mM sodium acetate, 200 mM sodium chloride, pH 5.5. Fractions containing the mixture of mono-, di- and tri-pegylated protein were pooled and characterized further.

### Example 13

Production, Purification and PEGylation of the [C17S+K19R+K33R+K45R+Q49N+Q51T+D110F+F111N+R113T] Human IFNB Variant in Roller Bottles.

A CHOK1 sub-clone (5/G-10) producing [C17S+K19R+K33R+K45R+Q49N+Q51T+D110F+F111N+R113T] human IFNB glycosylation variant was produced in 6 roller bottles as described in example 12 and purified according to the protocol used in example 12. The purity of the fully glycosylated [C17S+K19R+K33R+K45R+Q49N+Q51T+D110F+F111N+R113T] human IFNB variant was judged to be higher than 95% based on SDS-PAGE.

Following purification the variant was PEGylated. A fresh stock solution of SCM-PEG (succinimidyl ester of carboxymethylated PEG from Shearwater, Ala., 12 kDa or 20 kDa) was prepared in ethanol before each experiment.

A protein solution of 0.1 mg/ml in 20 mM sodium phosphate, pH 7.0 was PEGylated with SCM-PEG, 20K, with 3 times molar surplus of PEG to possible PEGylation sites, i.e. lysines and N-terminus. After incubation for 30 min at room termperature, the reaction was quenched by addition of a surplus of 20 mM glycine, pH 8.0. The reaction mixture contained a mixture of mono-, di- and un-pegylated material. Mono-pegylated material was separated from other species using either cation exchange chromatography or size-exclusion chromatography or a combination of both. pH in the PEGylation solution was adjusted to pH 2.7 and the sample was applied on a SP-Sepharose HR (Pharmacia) column equilibrated with 20 mM sodium citrate, pH 2.7. The pegylated protein was eluted from the column with 50 mM sodium acetate containing 1 M sodium chloride and applied on a size-exclusion column, Sephacryl S-100, ((16/60) Pharmacia) equilibrated with 100 mM sodium acetate, 200 mM sodium chloride, pH 5.5. Fractions containing mono-pegylated material was pooled and characterized further

In another experiment a protein solution of 0.1 mg/ml in 20 mM sodium phosphate, pH 7.0 was PEGylated with (10 mg/ml) SCM-PEG, 12K, with 5 times molar surplus of PEG to possible PEGylation sites, i.e. lysines and N-terminus. After incubation for 30 min at room termperature, the reaction was quenched by addition of a surplus of 20 mM glycine, pH 8.0. The reaction mixture contained a mixture of mono-, di-, tri-pegylated material together with underivatized material. The pegylated material was separated from the unmodified protein using either cation exchange chromatography or size-exclusion chromatography or a combination of both. pH in the PEGylation solution was adjusted to pH 2.7 and the sample was applied on a SP-Sepharose HR (Pharmacia) column equilibrated with 20 mM sodium citrate, pH 2.7. The pegylated protein was eluted from the column with 50 mM sodium acetate containing 1 M sodium chloride and applied on a size-exclusion column, Sephacryl S-100, ((16/60) Pharmacia) equilibrated with 100 mM sodium acetate, 200 mM sodium chloride, pH 5.5. Fractions containing the mixture of mono-, di- and tri-pegylated protein were pooled and characterized further.

1.-7. (canceled)

8. The method according to claim 39 or 44, wherein said glycosylation site is an in vivo N-glycosylation site.

9. The method according to claim 8, wherein the IFNB variant is asialo-glycosylated.

10. The method according to claim 39 or 44, wherein the amino acid sequence of said variant differs from the amino acid sequence of wild-type human IFNB (SEQ ID NO:2) in 1-15 amino acid residues.

11. The method according to claim 39 or 44, wherein said at least one glycosylation site is introduced by a substitution selected from the group consisting of S2N+N4T/S, L9N+R11T/S, R11N, S12N+N14T/S, F15N+C17S/T, Q16N+Q18T/S, K19N+L21T/S, Q23N+H25T/S, G26N+L28T/S, R27N+E29T/S, L28N+Y30T/S, D39T/S, K45N+L47T/S, Q46N+Q48T/S, Q48N+F50T/S, Q49N+Q51T/S, Q51N+E53T/S, R71N+D73T/S, Q72N, D73N, S75N, S76N+G78T/S, L88T/S, Y92T/S, N93N+195T/S, L98T/S, E103N+K105T/S, E104N+L106T/S, E107N+E109T/S, K108N+D110T/S, D110N, F111N+R113T/S and L116N.

12. The method according to claim 11, wherein said substitutions are selected from the group consisting of S2N+N4T, L9N+R11T, Q49N+Q51T, R71N+D73T and F111N+R113T.

13. The method according to claim 12, wherein said substitutions are selected from the group consisting of 4Q9N+Q51T, R71N+D73T and F111N+R113T.

14. The method according to claim 13, wherein said substitutions are selected from the group consisting of Q49N+Q51T and F111N+R113T.

15. The method according to claim 11, wherein said variant comprises substitutions selected from the group consisting of
Q49N+Q51T+F111N+R113T,
Q49N+Q51T+R71N+D73T+F111N+R113T,
S2N+N4T+F111N+R113T,
S2N+N4T+Q49N+Q51T,
S2N+N4T+Q49N+Q51T+F111N+R113T,
S2N+N4T+L9N+R11T+Q49N+Q51T,
S2N+N4T+L9N+R11T+F111N+R113T,
S2N+N4T+L9N+R11T+Q49N+Q51T+F111N+R113T,
L9N+R11T+Q49N+Q51T,
L9N+R11T+Q49N+Q51T+F111N+R113T and
L9N+R11T+F111N+R113T.

16. The method according to claim 15, wherein said variant comprises the substitutions Q49N+Q51T+F111N+R113T.

17. The method according to claim 39 or 44, wherein the cysteine residue located at position 17 in human wild-type IFNB (SEQ ID NO:2) has been removed.

18. The method according to claim 17, wherein said cysteine residue has been removed by the substitution C17S.

19. The method according to claim 18, wherein said variant comprises substitutions selected from the group consisting of
C17S+Q49N+Q51T,
C17S+F111N+R113T,
C17S+Q49N+Q51T+F111N+R113T,
C17S+Q49N+Q51T+R71N+D73T+F111N+R113T,
S2N+N4T+C17S+F111N+R113T,
S2N+N4T+C17S+Q49N+Q51T,
S2N+N4T+C17S+Q49N+Q51T+F111N+R113T,
S2N+N4T+L9N+R11T+C17S+Q49N+Q51T,
S2N+N4T+L9N+R11T+C17S+F111N+R113T,
S2N+N4T+L9N+R11T+C17S+Q49N+Q51T+F111N+R113T,
L9N+R11T+C17S+Q49N+Q51T,
L9N+R11T+C17S+Q49N+Q51T+F111N+R113T and
L9N+R11T+C17S+F111N+R113T.

20. The method according to claim 19, wherein said variant comprises the substitutions C17S+Q49N+Q51T+F111N+R113T.

21. The method according to claim 39 or 44, wherein said variant comprises a substitution in position 110.

22. The method according to claim 21, wherein said substitution is selected from the group consisting of D110F, D110V, D110W and D110Y.

23. The method according to claim 22, wherein said substitution is D110F.

24. The method according to claim 23, wherein said variant comprises substitutions selected from the group consisting of
C17S+D110F+F111N+R113T,
C17S+Q49N+Q51T+D110F+F111N+R113T,
C17S+Q49N+Q51T+R71N+D73T+D110F+F111N+R113T,
S2N+N4T+C17S+D110F+F111N+R113T,
S2N+N4T+C17S+Q49N+Q51T+D110F+F111N+R113T,
S2N+N4T+L9N+R11T+C17S+D110F+F111N+R113T,
S2N+N4T+L9N+R11T+C17S+Q49N+Q51T+D110F+F111N+R113T,
L9N+R11T+C17S+Q49N+Q51T+D110F+F111N+R113T and
L9N+R11T+C17S+D110F+F111N+R113T.

25. The method according to claim 24, wherein said variant comprises the substitutions C17S+Q49N+Q51T+D110F+F111N+R113T.

26. The method according to claim 25, wherein said variant has the amino acid sequence shown in SEQ ID NO:3.

27. The method according to claim 39 or 44, wherein a polymer molecule is covalently attached to an amino acid residue of the variant, said amino acid residue comprising an attachment group for the polymer molecule.

28. The method according to claim 27, wherein said polymer is a PEG molecule.

29. The method according to claim 27, wherein said attachment group is the ε-amino group of a lysine residue or the N-terminal amino group.

30. The method according to claim 27, wherein at least one lysine residue has been removed.

31. The method according to claim 30, wherein said lysine residue is selected from the group consisting of K19, K33, K45, K52, K99, K105, K108, K115, K123, K134 and K136.

32. The method according to claim 31, wherein said lysine residue is selected from the group consisting of K19, K33, K45 and K123.

33. The method according to claim 30, wherein said lysine residue has been removed by substituting said lysine residue with an arginine or glutamine residue.

34. The method according to claim 33, wherein said substitution(s) is (are) selected from the group consisting of K19R, K33R, K45R, K123R, K19R+K33R, K19R+K45R, K19R+K123R, K33R+K45R, K33R+K123R, K45R+K123R, K19R+K45R+K123R, K19R+K33R+K123R, K19R+K33R+K45R, K33R+K45R+K123R and K19R+K33R+K45R+K123R.

35. The method according to claim 34, wherein said substitutions are selected from the group consisting of K19R+K45R+K123R, K19R+K33R+K123R, K19R+K33R+K45R and K33R+K45R+K123R.

36. The method according to claim 35, wherein said substitutions are selected from the group consisting of K19R+K33R+K45R.

37. The method according to claim 36, wherein said variant comprises substitutions selected from the group consisting of
C17S+Q49N+Q51T+K19R+K33R+K45R,
C17S+D110F+F111N+R113T+K19R+K33R+K45R,
C17S+Q49N+Q51T+D110F+F111N+R113T+K19R+K33R+K45R,
C17S+Q49N+Q51T+R71N+D73T+D110F+F111N+R113T+K19R+K33R+K45R,
S2N+N4T+C17S+D1110F+F111N+R113T+K19R+K33R+K45R,
S2N+N4T+C17S+Q49N+Q51T+D110F+F111N+R113T+K19R+K33R+K45R,
S2N+N4T+L9N+R11T+C17S+D110F+F111N+R113T+K19R+K33R+K45R,
S2N+N4T+L9N+R11T+C17S+Q49N+Q51T+D110F+F111N+R113T+K19R+K33R+K45R,
L9N+R11T+C17S+Q49N+Q51T+D110F+F111N+R113T+K19R+K33R+K45R and
L9N+R11T+C17S+D110F+F111N+R113T+K19R+K33R+K45R.

38. The method according to claim 37, wherein said variant comprises the substitutions C17S+Q49N+Q51T+D110F+F111N+R113T+K19R+K33R+K45R.

39. A method for treating or preventing stroke or cerebrovascular accident (CVA) in a primate, the method comprising administering an effective amount of an interferon β (IFNB) polypeptide variant comprising an amino acid sequence which differs from the amino acid sequence of wild-type human IFNB (SEQ ID NO:2) in that at least one glycosylation site has been introduced, to a primate in need thereof.

40. The method according to claim 39, wherein said stroke is ischemic stroke.

41. The method according to claim 40, wherein said ischemic stroke is selected from the group consisting of embolic stroke, cardioembolic stroke, thrombotic stroke, large vessel thrombosis, lacunar infarction, artery-artery stroke and cryptogenic stroke.

42. The method according to claim 39, wherein said stroke is hemorrhagic stroke.

43. The method according to claim 42, wherein said hemorrhagic stroke is selected from the group consisting of intraparenchymal stroke, subdural stroke, epidural stroke and subarachnoid stroke.

44. A method for treating or preventing transient ischemic attack in a primate, the method comprising administering an effective amount of an interferon β (IFNB) polypeptide variant comprising an amino acid sequence which differs from the amino acid sequence of wild-type human IFNB (SEQ ID NO:2) in that at least one glycosylation site has been introduced, to a primate in need thereof.

45. The method according to claim 39 or 44, wherein said primate is a human.

46. (canceled)


SEQUENCE LISTING (51 sequences)

SEQ ID NO: 1 - DNA, 840 bp, Artificial Sequence
  Description of Artificial Sequence Expression casette for expression of IFNB in mammalian and insect cells
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 2 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - protein, 166 aa, Artificial Sequence
  Description of Artificial Sequence IFNB variant
MSYNLLGFLQRSSNFQSQKLLWQLNGRLEYCLKDRMNFDIPEEIKQLQNFTKEDAALTIYEMLQNIFAIFRQDSSSTGWNETIVENLLANVYHQINHLKTVLEEKLEKEFNTTGKLMSSLHLKRYYGRILHYLKAKEYSHCAWTIVRVEILRNFYFINRLTGYLRN

SEQ ID NO: 4 - DNA, 70 bp, Artificial Sequence
  Description of Artificial Sequence primer
ggctagcgtttaaacttaagcttcgccaccatgaccaacaagtgcctgctccagatcgccctgctcctgt

SEQ ID NO: 5 - DNA, 70 bp, Artificial Sequence
  Description of Artificial Sequence primer
acaacctgctcggcttcctgcagaggagttcgaacttccagtgccagaagctcctgtggcagctgaacgg

SEQ ID NO: 6 - DNA, 70 bp, Artificial Sequence
  Description of Artificial Sequence primer
gaacttcgacatccccgaggaaatcaagcagctgcagcagttccagaaggaggacgccgctctgaccatc

SEQ ID NO: 7 - DNA, 70 bp, Artificial Sequence
  Description of Artificial Sequence primer
ttccgccaggactccagctccaccggttggaacgagaccatcgtggagaacctgctggccaacgtgtacc

SEQ ID NO: 8 - DNA, 70 bp, Artificial Sequence
  Description of Artificial Sequence primer
aggagaagctggagaaggaggacttcacccgcggcaagctgatgagctccctgcacctgaagcgctacta

SEQ ID NO: 9 - DNA, 70 bp, Artificial Sequence
  Description of Artificial Sequence primer
ggagtacagccactgcgcctggaccatcgtacgcgtggagatcctgcgcaacttctacttcatcaaccgc

SEQ ID NO: 10 - DNA, 70 bp, Artificial Sequence
  Description of Artificial Sequence primer
caccacactggactagtggatccttatcagttgcgcaggtagccggtcaggcggttgatgaagtagaagt

SEQ ID NO: 11 - DNA, 70 bp, Artificial Sequence
  Description of Artificial Sequence primer
aggcgcagtggctgtactccttggccttcaggtagtgcaggatgcggccatagtagcgcttcaggtgcag

SEQ ID NO: 12 - DNA, 70 bp, Artificial Sequence
  Description of Artificial Sequence primer
ctccttctccagcttctcctccagcacggtcttcaggtggttgatctggtggtacacgttggccagcagg

SEQ ID NO: 13 - DNA, 70 bp, Artificial Sequence
  Description of Artificial Sequence primer
gagctggagtcctggcggaagatggcgaagatgttctgcagcatctcgtagatggtcagagcggcgtcct

SEQ ID NO: 14 - DNA, 70 bp, Artificial Sequence
  Description of Artificial Sequence primer
cctcggggatgtcgaagttcatcctgtccttcaggcagtactccaggcgcccgttcagctgccacaggag

SEQ ID NO: 15 - DNA, 70 bp, Artificial Sequence
  Description of Artificial Sequence primer
caggaagccgagcaggttgtagctcatcgatagggccgtggtgctgaagcacaggagcagggcgatctgg

SEQ ID NO: 16 - DNA, 70 bp, Artificial Sequence
  Description of Artificial Sequence primer
ctgctccagatcgccctgctcctgtgcttcagcaccacggccctatcgatgaagcaccagcaccagcatc

SEQ ID NO: 17 - DNA, 70 bp, Artificial Sequence
  Description of Artificial Sequence primer
cactgcttactggcttatcgaaattaatacgactcactatagggagacccaagctggctagcgtttaaac

SEQ ID NO: 18 - DNA, 70 bp, Artificial Sequence
  Description of Artificial Sequence primer
caggaagccgagcaggttgtagctcatctgttggtgttgatgttggtgctgatgctggtgctggtgcttc

SEQ ID NO: 19 - DNA, 70 bp, Artificial Sequence
  Description of Artificial Sequence primer
agcagggcgatctggagcaggcacttgttggtcatggtggcgaagcttaagtttaaacgctagccagctt

SEQ ID NO: 20 - DNA, 40 bp, Artificial Sequence
  Description of Artificial Sequence primer
ccgtcagatcctaggctagcttattgcggtagtttatcac

SEQ ID NO: 21 - DNA, 32 bp, Artificial Sequence
  Description of Artificial Sequence primer
gagctcggtaccaagcttttaagagctgtaat

SEQ ID NO: 22 - DNA, 77 bp, Artificial Sequence
  Description of Artificial Sequence primer
gctgaacgggcgcctggagtactgcctgaaggacaggatgaacttcgacatccccgaggaaatccgccagctgcagc

SEQ ID NO: 23 - DNA, 35 bp, Artificial Sequence
  Description of Artificial Sequence primer
tctccacgcgtacgatggtccaggcgcagtggctg

SEQ ID NO: 24 - DNA, 70 bp, Artificial Sequence; identical to SEQ ID NO: 10
  Description of Artificial Sequence primer

SEQ ID NO: 25 - DNA, 31 bp, Artificial Sequence
  Description of Artificial Sequence primer
catcagcttgccggtggtgttgtcctccttc

SEQ ID NO: 26 - DNA, 31 bp, Artificial Sequence
  Description of Artificial Sequence primer
gaaggaggacaacaccaccggcaagctgatg

SEQ ID NO: 27 - DNA, 40 bp, Artificial Sequence
  Description of Artificial Sequence primer
cacactggactagtaagcttttatcagttgcgcaggtagc

SEQ ID NO: 28 - DNA, 47 bp, Artificial Sequence
  Description of Artificial Sequence primer
gaggagttcgaacttccagtgccagcgcctcctgtggcagctgaacg

SEQ ID NO: 29 - DNA, 33 bp, Artificial Sequence
  Description of Artificial Sequence primer
tttaaactggatccagccaccatgaccaacaag

SEQ ID NO: 30 - DNA, 63 bp, Artificial Sequence
  Description of Artificial Sequence primer
cggccatagtagcgcttcaggtgcagggagctcatcagcttgccggtggtgttgtcctccttc

SEQ ID NO: 31 - DNA, 63 bp, Artificial Sequence
  Description of Artificial Sequence primer
gaaggaggacaacaccaccggcaagctgatgagctccctgcacctgaagcgctactatggccg

SEQ ID NO: 32 - DNA, 30 bp, Artificial Sequence
  Description of Artificial Sequence primer
ggcgtcctccttggtgaagttctgcagctg

SEQ ID NO: 33 - DNA, 39 bp, Artificial Sequence
  Description of Artificial Sequence primer
atatatcccaagcttttatcagttgcgcaggtagccggt

SEQ ID NO: 34 - DNA, 30 bp, Artificial Sequence
  Description of Artificial Sequence primer
cagctgcagaacttcaccaaggaggacgcc

SEQ ID NO: 35 - DNA, 34 bp, Artificial Sequence
  Description of Artificial Sequence primer
cgcggatccagccaccatgaccaacaagtgcctg

SEQ ID NO: 36 - DNA, 34 bp, Artificial Sequence; identical to SEQ ID NO: 35
  Description of Artificial Sequence primer

SEQ ID NO: 37 - DNA, 30 bp, Artificial Sequence
  Description of Artificial Sequence primer
gtcctccttggtgaagttgaacagctgctt

SEQ ID NO: 38 - DNA, 39 bp, Artificial Sequence; identical to SEQ ID NO: 33
  Description of Artificial Sequence primer

SEQ ID NO: 39 - DNA, 30 bp, Artificial Sequence
  Description of Artificial Sequence primer
aagcagctgttcaacttcaccaaggaggac

SEQ ID NO: 40 - DNA, 34 bp, Artificial Sequence; identical to SEQ ID NO: 35
  Description of Artificial Sequence primer

SEQ ID NO: 41 - DNA, 30 bp, Artificial Sequence
  Description of Artificial Sequence primer
gtcctccttggtgaagttcaccagctgctt

SEQ ID NO: 42 - DNA, 30 bp, Artificial Sequence
  Description of Artificial Sequence primer
aagcagctggtgaacttcaccaaggaggac

SEQ ID NO: 43 - DNA, 34 bp, Artificial Sequence; identical to SEQ ID NO: 35
  Description of Artificial Sequence primer

SEQ ID NO: 44 - DNA, 30 bp, Artificial Sequence
  Description of Artificial Sequence primer
gtcctccttggtgaagttccacagctgctt

SEQ ID NO: 45 - DNA, 30 bp, Artificial Sequence
  Description of Artificial Sequence primer
aagcagctgtggaacttcaccaaggaggac

SEQ ID NO: 46 - DNA, 34 bp, Artificial Sequence; identical to SEQ ID NO: 35
  Description of Artificial Sequence primer

SEQ ID NO: 47 - DNA, 30 bp, Artificial Sequence
  Description of Artificial Sequence primer
cagcttgccggtggtgttgaactccttctc

SEQ ID NO: 48 - DNA, 30 bp, Artificial Sequence
  Description of Artificial Sequence primer
gagaaggagttcaacaccaccggcaagctg

SEQ ID NO: 49 - DNA, 34 bp, Artificial Sequence; identical to SEQ ID NO: 35
  Description of Artificial Sequence primer

SEQ ID NO: 50 - DNA, 30 bp, Artificial Sequence
  Description of Artificial Sequence primer
cagcttgccggtggtgttcacctccttctc

SEQ ID NO: 51 - DNA, 30 bp, Artificial Sequence
  Description of Artificial Sequence primer
gagaaggaggtgaacaccaccggcaagctg